(19) United States
(12) Patent Application Publication
(10) Pub. No.: US 2025/0228756 A1
(43) Pub. Date: Jul. 17, 2025

(54) Anhydrous Urea Emulsions with a Retinoid Agent

(71) Applicant: Baek Clinical Inc., Los Angeles, CA (US)

(72) Inventor: Jihoon P. Baek, Los Angeles, CA (US)

(21) Appl. No.: 18/703,024

(22) PCT Filed: Oct. 20, 2022
(86) PCT No.: PCT/US2022/047305

§ 371 Date: Apr. 19, 2024

Related U.S. Application Data
(60) Provisional application No. 63/257,672, filed on Oct. 20, 2021.

(51) Int. Cl. A61K 8/42; A61K 8/06; A61K 8/34; A61K 8/67; A61Q 19/00

(52) U.S. Cl. CPC A61K 8/42; A61K 8/06; A61K 8/345; A61K 8/671; A61K 8/676; A61K 8/678; A61Q 19/00

(57) ABSTRACT

Topical formulations of urea and a retinoid agent dissolved in a non-aqueous skin-compatible solvent and combined with organosilicone compounds into an emulsion are provided. The formulations are storage stable for an extended period of time without significant degradation of the urea or retinoid agent in the composition and have desirable physical properties. Topical compositions of this disclosure find use in treating or preventing a variety of cosmetic and/or dermatological conditions.

### CROSS-REFERENCE TO RELATED APPLICATIONS

This claims the benefit of U.S. Provisional Patent Application No. 63/257,672, filed Oct. 20, 2021, which application is incorporated herein by reference in its entirety

### FIELD OF INVENTION

Topical compositions and methods for treating, preventing, or improving dermatocosmetic conditions.

### INTRODUCTION

Urea (also commonly referred to as carbamide) is a potent humectant, emollient and keratolytic agent, and is widely used in topical compositions to treat or prevent a range of cosmetic and/or dermatological conditions associated with dry and scaly skin, such as dermatitis, psoriasis, xerosis, ichthyosis, eczema, keratosis, and keratosis pilaris. M Pan et al., Dermatology Online Journal Vol. 19 (2013).

Studies suggest that the keratolytic and hydrating effects of topical urea is owing to breakage of hydrogen bonds in the stratum corneum, loosening epidermal keratin and increasing water-binding sites. M Gloor et al., Skin Pharmacol. Appl. Skin Physiol. Vol. 15 (2002). In topical compositions, the use of urea (and substituted ureas) is well known, including for moisture retention (as a humectant), for keratolytic activity, as well as for penetration enhancement, both for itself and other active ingredients. At concentrations of lower than about 10%, urea acts as a moisturizer. At higher concentrations, from about 10% up to 40%, urea can be used to treat dry/rough skin conditions, including ichthyosis and psoriasis.

Inclusion of urea at efficacious concentrations in aqueous topical compositions poses formulation challenges. Urea can undergo steady hydrolysis, producing ammonia and other amine byproducts, compounds that not only have an unpleasant odor but also tend to increase pH. Moreover, hydrolysis of urea in aqueous compositions can cause discoloration or other breakdown of the product composition, including phase separation.

The byproducts of urea decomposition pose two main issues: (1) a constant rise in pH that can adversely affect the formula's stability and preservative efficacy over time, and (2) the release of an unpleasant odor due to the escape of ammonia.

Research and development activities seeking to mitigate the effects of urea decomposition have focused on pH optimization and stabilization via the usage of pH buffer systems (e.g., including acetic acid, lactic acid, lactones, etc.), neutralization of the ammonia produced by urea breakdown, and the use of packaging components that prevent the escape of ammonia and carbon dioxide byproducts. RP Raab, J Appl Cosmetol. Vol. 9 (1991).

Retinoids are a class of compounds that are vitamers of vitamin A, or are chemically related to vitamin A. Examples of retinoids include, retinol, retinal, tretinoin (all-trans-retinoic acid), retinol esters (such as retinyl acetate, retinyl propionate etc.), isotretinoin, alitretinoin, etretinate, acitretin, adapalene, bexarotene, and tazarotene. Retinol (vitamin A) is an endogenous compound which occurs naturally in the human body, and is essential for normal epithelial cell differentiation. Natural and synthetic vitamin A derivatives have been used extensively in the treatment of a variety of skin disorders and have been used as skin repair or renewal agents. Retinoic acid has been employed to treat a variety of skin conditions, e.g., acne, wrinkles, psoriasis, age spots and discoloration. See e.g., Vahlquist, A. et al., J. Invest. Dermatol., Vol. 94, Holland D. B. and Cunliffe, W. J. (1990), pp. 496-498; Ellis, C. N. et al., “Pharmacology of Retinols in Skin”, Vasel, Karger, Vol. 3, (1989), pp. 249-252; Lowe, N.J. et al., “Pharmacology of Retinols in Skin”, Vol. 3, (1989), pp. 240-248; PCT Publication No. WO 93/19743.

Inclusion of a retinoid agent at efficacious concentrations in topical compositions can also pose formulation challenges. For example, retinoid agents such as retinol can undergo oxidation readily to result in products which have no effect on the skin. Further, a common side effect of retinoid agents (e.g., retinol) in topical formulations is skin irritation. For example, when one commences use of a topical retinoid containing formulation, one may experience skin irritation during a period when the skin is adjusting to the retinoid agent (a process generally known as “retinization”).

There has been and remains a need for non-gritty, non-oily/non-greasy, non-irritating topical formulations that contain and maintain an efficacious concentration of components without degradation, and concomitant decrease in activity. There is also a need for topical formulations including a retinoid agent which reduce irritation to sensitive skin.

### SUMMARY

Topical formulations of an anhydrous emulsion, composed of a urea compound, a retinoid agent, non-aqueous skin-compatible solvents, and silicone compound are provided. The formulations are storage stable in a non-aqueous solution for an extended period of time without significant degradation of the urea or the retinoid agent in the composition and have desirable physical properties. The topical formulations can include concentrations of urea of 1 to 30% by weight and a retinoid agent in a concentration of 0.01 to 1.5% by weight. Topical compositions of this disclosure find use in treating or preventing a variety of cosmetic and/or dermatological conditions.

Polyol in silicone emulsions are challenging and uncommon to make stable. The inventor of the present disclosure surprisingly generated a polyol in silicone emulsion formulation with urea that was storage stable. Additionally, even with a formulation that includes a retinoid agent, the present inventor found that the formulations of the present disclosure were stable.

An aspect of the present disclosure includes a storage stable topical emulsion composition comprising: (a) a dispersed phase that is a homogenous solution comprising: 1% to 30% by weight of urea agent; dissolved in 10% or more by weight of a non-aqueous skin-compatible solvent selected from polyol, C₍₁₋₆₎ alkanediol, glycol ether, dimethyl ether, and a combination thereof; and (b) a continuous phase comprising: 0.01 to 1.5% of a retinoid agent; and 10% or more by composition weight of a silicone agent selected from cyclic, linear and branched silicones, a silicone crosspolymer, and a combination thereof, wherein the dispersed phase is immiscible with and contained within the continuous phase.

In some embodiments, the emulsion composition further comprises an antioxidant. In some embodiments, the antioxidant is selected from ascorbic acid, and azelaic acid, and any combination thereof.

In some embodiments, the composition comprises ascorbic acid. In some embodiments, the composition comprises 22% or less by weight of ascorbic acid dissolved in the dispersed phase or the continuous phase. In some embodiments, the composition comprises about 5% to 15% by weight of ascorbic acid dissolved in the dispersed phase or the continuous phase. In some embodiments, the composition comprises about 5% by weight of ascorbic acid dissolved in the dispersed phase or the continuous phase.

In some embodiments, the composition comprises azelaic acid. In some embodiments, the composition comprises 22% or less by weight of azelaic acid dissolved in the dispersed phase or the continuous phase. In some embodiments, the composition comprises about 5% to 15% by weight of azelaic acid dissolved in the dispersed phase or the continuous phase. In some embodiments, the composition comprises about 5% by weight of azelaic acid dissolved in the dispersed phase or the continuous phase.

In some embodiments, the composition comprises 0.015 to 1% by weight of a retinoid agent. In some embodiments, the composition comprises 0.02% by weight of retinoid agent. In some embodiments, the composition comprises 0.015 to 1.5% by weight of a retinoid agent. In some embodiments, the retinoid agent is in the continuous phase.

In some embodiments, the composition comprises 0.5% by weight of ferulic acid.

In some embodiments, the retinoid agent is selected from retinol, a retinyl ester, retinal, tretinoin, isotretinoin, alitretinoin, etretinate, acitretin, adapalene, bexarotene and tazarotene. In some embodiments, the retinoid agent is retinol. In some embodiments, the retinoid agent is a retinyl ester (e.g., retinyl acetate, or retinyl propionate) In some embodiments, the retinoid agent is tretinoin. In some embodiments, the retinoid is adapalene or tararotene.

In some embodiments, the continuous phase is a solution of the silicone agent in an oil-phase. In some embodiments, the silicone agent is selected from dimethicone, PEG-10/15 crosspolymer, dimethicone/polyethylene glycol (PEG)-10/15 crosspolymer, lauryl PEG-9 polydimethylsiloxyethyl dimethicone, and a combination thereof.

In some embodiments, the composition comprises 5 to 20% by weight of the silicone agent. In some embodiments, the urea agent is urea. In some embodiments, the urea agent is hydroxyethyl urea. In some embodiments, the urea agent comprises a mixture of urea and hydroxyethyl urea.

In some embodiments, the non-aqueous solvent is selected from 1,3 propanediol, 1,2 propanediol, 1,3 butanediol, 1,5 pentanediol, 1,2 hexanediol, 1,6 hexanediol, glycerol, diglycerol, ethoxydiglycol, dimethyl isosorbide and a combination thereof. In some embodiments, the solvent is 1,3 propanediol.

In some embodiments, the composition exhibits a urea degradation rate that is less than the urea degradation rate of a homogenous dispersed phase solution in the absence of the continuous phase emulsion. In some embodiments, the composition comprises 20% to 80% by weight of the silicone agent of the continuous phase. In some embodiments, the composition comprises 5% to 20% by weight of the urea agent.

In some embodiments, the composition comprises 0.015% to 0.5% by weight of the retinoid agent. The composition of any one of claims 1-31, wherein the percent by weight ratio of the dispersed phase to the continuous phase in the composition is 19 or less. In some embodiments, the composition further comprises 10% or less by weight in total of one or more optional additional components dissolved in the dispersed and/or continuous phase (e.g., additional antioxidants, soothing agents, etc.). In some embodiments, the one or more optional additional components are selected from tocopherols, tocotrienols (e.g., alpha, beta, delta and gamma tocopherols or alpha, beta, delta and gamma tocotrienols), cinnamic acid, ferulic acid, hydroxy acids (e.g., salicylic acid), panthenol, Pinus pinaster bark extract, hyaluronic acid complex, cholesterol ester, cholesterol, ceramide, linoleic acid, linolenic acid, madecassoside, acetyl zingerone, bakuchiol, bis-ethylhexyl hydroxydimethoxy benzylmalonate, zinc oxide, and titanium dioxide

In some embodiments, the composition comprises about 5% to about 20% by weight of urea. In some embodiments, the composition comprises about 5% by weight of urea. In some embodiments, the composition comprises about 10% by weight of urea. In some embodiments, the optional additional component comprises ferulic acid. In some embodiments, the composition comprises 0.1% to 2% by weight of ferulic acid. In some embodiments, the composition comprises 0.5% by weight of ferulic acid.

In some embodiments, the optional additional component comprises vitamin E. In some embodiments, the vitamin E is selected from alpha, beta, delta and gamma tocopherols and alpha, beta, delta and gamma tocotrienols, and combinations thereof.

In some embodiments, the composition comprises 2% or less by weight of vitamin E. In some embodiments, the optional additional component is bis-ethylhexyl hydroxydimethoxy benzylmalonate. In some embodiments, the composition comprises 2% or less by weight of bis-ethylhexyl hydroxydimethoxy benzylmalonate.

In some embodiments, the optional additional component comprises bakuchiol. In some embodiments, the composition comprises 2% or less of bakuchiol. In some embodiments, the optional additional component comprises C10-C30 cholesterol/lanosterol esters. In some embodiments, the composition comprises 5% or less of C10-C30 cholesterol/lanosterol esters.

In some embodiments, the optional additional component comprises madecassoside, asiaticoside, or a combination thereof. In some embodiments, the composition comprises 1% or less by weight of a combination of madecassoside and asiaticoside. In some embodiments, the optional additional component comprises glycyrrhetinic acid. In some embodiments, the composition comprises 1% or less of glycyrrhetinic acid.

In some embodiments, the optional additional component comprises Pinus pinaster bark extract. In some embodiments, the composition comprises 0.5% to 2% by weight of Pinus pinaster bark extract.

In some embodiments, the optional additional component comprises a ceramide. In some embodiments, the ceramide is selected from ceramide EOP, ceramide AP, ceramide NG, ceramide NP, ceramide NS, ceramide EOS, ceramide S, ceramide AS, and combinations thereof.

In some embodiments, the composition comprises 2% or less by weight of ceramide.

In some embodiments, the optional additional component comprises cholesterol. In some embodiments, the composition comprises less than 2% by weight of cholesterol.

In some embodiments, the optional additional component comprises a free fatty acid. In some embodiments, the free fatty acid is selected from linoleic acid, linolenic acid, stearic acid, palmitic acid, oleic acid, alpha-linoleic, oleic acid, and combinations thereof. In some embodiments, the composition comprises less than 1% free fatty acid.

In some embodiments, droplets of the dispersed phase are contained within the continuous phase. In some embodiments, the composition comprises: in the dispersed phase: 56% by weight of propanediol; 5% by weight of urea; 5% by weight of azelaic acid; and 5% by weight of ascorbic acid; and in the continuous phase: 0.02% by weight of the retinoid agent.

In some embodiments, the composition comprises: in the dispersed phase: 56% by weight of propanediol; 5% by weight of urea; 5% by weight of azelaic acid; and 5% by weight of ascorbic acid; and in the continuous phase: 1% by weight of a combination of Allyl Methacrylate Crosspolymer, Polysorbate 20, Retinol and Tocopherol; 4% by weight of C10-30 cholesterol/lanosterol esters; 0.5% by weight of Bakuchiol; 1% by weight of Tocopherol; and 20% by weight of dimethicone and dimethicone/PEG-10/15 crosspolymer.

In some embodiments, the composition comprises: in the dispersed phase: 56% by weight of propanediol; 5% by weight of urea; 5% by weight of azelaic acid; and 5% by weight of ascorbic acid; and in the continuous phase: 0.2% by weight of the retinoid agent; 0.8% by weight of a combination of Allyl Methacrylate Crosspolymer, Polysorbate 20, and Tocopherol; 4% by weight of C10-30 cholesterol/lanosterol esters; 0.5% by weight of Bakuchiol; 1% by weight of Tocopherol; and 20% by weight of dimethicone and dimethicone/PEG-10/15 crosspolymer.

Another aspect of the present disclosure includes a ready-to-use topical preparation in a multi-use container which is pre-filled with a storage stable topical composition described herein, wherein the multi-use container comprises means for dispensing a single dose of the storage stable topical composition.

In some embodiments, the multi-use container is a time-release container that delivers urea and the retinoid agent consistently over a period of time. In some embodiments, the time-release container does not release urea and the retinoid agent all at once. In some embodiments, the storage stable topical composition is sealed in the container. In some embodiments, the container is placed in packaging.

Another aspect of the present disclosure includes a process for producing a storage stable emulsion composition for topical application, the process comprising: (a) combining: 1% to 30% by weight of urea agent selected from urea, hydroxyethyl urea, and combination thereof; 10% to 80% by weight of a non-aqueous skin-compatible solvent selected from polyol, C₍₁₋₆₎ alkanediol, glycol ether, dimethyl ether, and a combination thereof; and optionally one or more additional agents; thereby dissolving the urea agent, and one or more additional agents in the non-aqueous solvent to produce a dispersed phase that is a homogenous solution; and suspending the dispersed phase in a continuous phase comprising 10% or more by weight of a silicone compound dissolved in an oil-phase solution; and 0.01% to 1.5% of a retinoid agent; to produce an emulsion composition that is storage stable.

In some embodiments, the silicone compound is selected from cyclic, linear and branched silicones, a silicone crosspolymer, and a combination thereof.

In some embodiments, the one or more additional agents comprise ascorbic acid. In some embodiments, the dispersed phase comprises 22% or less by weight of ascorbic acid. In some embodiments, the dispersed phase comprises about 5% to about 15% by weight of ascorbic acid.

In some embodiments, the one or more additional agents comprise ferulic acid. In some embodiments, the composition comprises 2% or less by weight of ferulic acid.

In some embodiments, the one or more additional agents comprise: 0.5% to 2% ferulic acid; and 0.5% to 2% Pinus pinaster bark extract.

In some embodiments, the one or more additional agents comprise about 3% to about 10% by weight azelaic acid. In some embodiments, the one or more additional agents comprise about 0.01% to 1.5% by weight of a retinoid agent. In some embodiments, the retinoid agent is in the continuous phase.

In some embodiments, the continuous phase further comprises a lipid component.

In some embodiments, the lipid component is selected from cholesterol, ceramides, free fatty acids, and combinations thereof. In some embodiments, the continuous phase prevents or reduces precipitation of urea out of the emulsion composition.

In some embodiments, the urea agent, prior to dissolving in the non-aqueous solvent, is a crystalline form of the urea agent with a particle size of about 100 μm or more. In some embodiments, suspending the dispersed phase in the continuous phase prevents or reduces recrystallization of urea from the dispersed phase. In some embodiments, suspending comprises suspending droplets of the dispersed phase within the continuous phase.

A product produced by the process described herein. In some embodiments, the product is a serum.

### DETAILED DESCRIPTION

This disclosure provides topical formulations of urea dissolved in a non-aqueous skin-compatible solvent, combined with a retinoid agent and a silicone agent as an emulsifying agent. The formulations are storage stable in a non-aqueous solution for an extended period of time without undesirable change in odor, significant oxidation of the retinoid agent, or significant degradation of the urea in the composition. This disclosure provides particular topical formulations which have been developed and optimized to provide skin compatibility and desirable physical properties.

Topical compositions of this disclosure find use in treating or preventing a variety of cosmetic and/or dermatological conditions. Non-limiting examples of dermatocosmetic conditions that may be improved by topical application of the compositions of the present disclosure include: inflammatory dermatoses (including eczema, acne, psoriasis), and xeroses (also known in the art as dry skin or pruritus).

In some embodiments, formulations of the present disclosure include the ingredients: (i) 1 to 30% by weight urea agent; dissolved in (ii) a non-aqueous skin-compatible solvent; combined with (iii) 0.01 to 1.5% of a retinoid agent, and (iv) a silicone agent to form an emulsion.

Hydrolysis of urea in aqueous compositions can cause discoloration or other breakdown of the product, including phase separation. The inventor of the present disclosure discovered that by incorporating a substantially anhydrous first phase (e.g., dispersed phase) containing urea and a non-aqueous solvent, the resulting first phase (e.g., dispersed phase) solubilizes urea by circumventing hydrolysis.

The inventor of the present disclosure discovered that by incorporating a homogenous non-aqueous solution of urea and optionally a retinoid agent into an emulsion, the resulting compositions provide for exclusion of moisture, preventing urea degradation. Urea undergoes steady hydrolysis, producing ammonia and other amines, compounds that not only have an unpleasant odor but also tend to increase pH. The present inventor discovered that an emulsion containing a homogenous non-aqueous solution of urea prevents discoloration and unpleasant odor caused by degradation of urea; and stabilizes the pH of the composition.

The inventor of the present disclosure surprisingly discovered that particular amounts of a urea agent and retinoid agent can be added to a non-aqueous solvent to provide stable solutions of urea agent and retinoid agent at various desired concentration levels.

The inventor of the present disclosure discovered that by incorporating a homogenous non-aqueous solution of urea and a retinoid agent into an emulsion, the resulting compositions reduce the skin irritation.

The inventor of the present disclosure discovered that by incorporating a homogenous non-aqueous solution of urea and a retinoid agent into an emulsion, the resulting compositions are storage stable in a non-aqueous solution.

### Urea Agent

Urea agents of interest include, but are not limited to, urea and substituted urea, such as alkyl substituted urea, more particularly mono-substituted or di-substituted alkyl urea (e.g., hydroxyalkyl urea). In some embodiments, the urea agent is a hydroxyalkyl urea, such as hydroxyethyl urea. The urea agent ingredient used in the subject formulations can be a combination of urea and/or substituted ureas. For example, the urea agent can be a combination of urea and hydroxyethyl urea. In certain embodiments, the urea agent is urea. In certain embodiments, the urea agent is hydroxyethyl urea.

In certain embodiments, the urea agent used in preparing the subject compositions is in a crystalline form before solubilized in a solvent. In some embodiments, the crystalline form of urea has a particle size (e.g., mean particle size) of 100 microns or more, 125 microns or more, 150 microns or more, 175 microns or more, 200 microns or more, 225 microns or more, 250 microns or more, 275 microns or more, or 300 microns or more.

In some embodiments, the amount of a urea agent in the subject composition is at least about 5% by weight, such as at least about 6% by weight, at least about 7% by weight, at least about 8% by weight, at least about 9% by weight, at least about 10% by weight, at least about 11% by weight, at least about 12% by weight, at least about 14% by weight, at least about 15% by weight, at least about 16% by weight, at least about 17% by weight, at least about 18% by weight, at least about 19% by weight, at least about 20% by weight, at least about 21% by weight, at least about 22% by weight, at least about 23% by weight, at least about 24% by weight, or at least about 25% by weight. In some embodiments, the subject composition includes about 28% by weight or less of a urea agent in the non-aqueous solvent solution, such as about 25% by weight or less.

In certain embodiments, the non-aqueous solvent is 1,3-propanediol. In particular embodiments, the amount of a urea agent in the subject composition is between about 10% by weight and about 20% by weight, or between about 12% by weight and about 28% by weight, such as between about 15% by weight and about 28% by weight, or between about 20% by weight and about 28% by weight. In some embodiments, the amount of a urea agent in the subject composition is about 5%, about 10%, about 15%, about 20%, or about 25% by weight.

In general, the amounts of urea agent in a composition are calculated relative to the solution phase based on the non-aqueous solvent. However, the amounts of urea agent and other ingredients relative to the emulsion composition as a whole can readily be calculated by the skilled artisan. It is understood that, in some cases, these concentrate solutions having particular amounts of urea agent can be combined with an immiscible ingredient (e.g., an oil component) and an emulsifying agent to produce an emulsion composition (e.g., as described below).

For example, for compositions including 15% by weight of a urea agent, at least about 4% urea is included in the 1,3-propanediol solvent. For compositions including 20% by weight a of a urea agent, at least about 10% urea is included in the 1,3-propanediol solvent. For compositions including 25% by weight of a urea agent, at least about 16% urea is included in the 1,3-propanediol solvent.

In some embodiments, the subject composition includes about 1 to 30% by weight of a urea agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and a non-aqueous solvent. In some embodiments, the subject composition includes between 5-10%, about 10-15%, or about 15-20% by weight of a urea agent, and a non-aqueous solvent. In certain embodiments, the subject composition includes about 5% by weight of a urea agent and a non-aqueous solvent. In certain embodiments, the subject composition includes about 15% by weight of a urea agent and a non-aqueous solvent. In certain embodiments, the subject composition includes about 20% by weight of a urea agent and a non-aqueous solvent.

In some embodiments, the subject composition includes about 5 to 7% by weight of a urea agent and a non-aqueous solvent. In some embodiments, the subject composition includes about 7 to 9% by weight of a urea agent and a non-aqueous solvent. In certain embodiments, the subject composition includes about 9 to 11% by weight of a urea agent and a non-aqueous solvent. In certain embodiments, the subject composition includes about 11 to 13% by weight of a urea agent and a non-aqueous solvent. In certain embodiments, the subject composition includes about 13 to 15% by weight of a urea agent and a non-aqueous solvent. In certain embodiments, the subject composition includes about 15 to 17% by weight of a urea agent and a non-aqueous solvent. In certain embodiments, the subject composition includes about 17 to 19% by weight of a urea agent and a non-aqueous solvent. In certain embodiments, the subject composition includes about 19 to 21% by weight of a urea agent and a non-aqueous solvent. In certain embodiments, the subject composition includes about 21 to 23% by weight of a urea agent and a non-aqueous solvent. In certain embodiments, the subject composition includes about 23 to 25% by weight of a urea agent and a non-aqueous solvent.

In some embodiments, a first phase (e.g., dispersed/polyol phase) of the subject composition includes about 5 to 50% by weight of a urea agent. In some embodiments, the first phase (e.g., dispersed/polyphase) of the subject composition includes the first phase (e.g., dispersed phase) comprising about 5% or more, about 6% or more, about 7% or more, about 8% or more, about 9% or more, about 10% or more, about 11% or more, about 12% or more, about 13% or more, about 14% or more, about 15% or more, about 16% or more, about 17% or more, about 18% or more, about 19% or more, about 20% or more, about 21% or more, about 22% or more, about 23% or more, about 24% or more, about 25% or more, about 26% or more, about 27% or more, about 28% or more, about 29% or more, about 30% or more, about 31% or more, about 32% or more, about 33% or more, about 34% or more, about 35% or more, about 36% or more, about 37% or more, about 38% or more, about 39% or more, about 40% or more, about 41% or more, about 42% or more, about 43% or more, about 44% or more, about 45% or more, about 46% or more, about 47% or more, about 48% or more, about 49% or more, or about 50% or more by weight of the urea agent.

In some embodiments, the dispersed phase can be the dispersed phase can be as high as 70-80%. Alternatively, in other embodiments, the dispersed phase can be as low as 20% and still reach a 5% urea level.

### Retinoid Agents

A retinoid agent is a compound of the class of retinoids, which includes compounds that are derivatives or analogs of vitamin A. Retinoid agents of interest include, but are not limited to, retinyl esters, retinol, retinal and retinoic acid. In certain cases, the retinoid is selected from retinol, retinal, tretinoin (all-trans-retinoic acid), a retinol ester, isotretinoin, alitretinoin, etretinate, acitretin, adapalene, bexarotene, and tazarotene. In certain cases, the retinoid is retinal or a retinyl ester. The term “retinol” includes the following isomers of retinol: all-trans-retinol, 13-cis-retinol, 11-cis-retinol, 9-cis-retinol, 3,4-didehydro-retinol, 3,4-didehydro-13-cis-retinol; 3,4-didehydro-11-cis-retinol; 3,4-didehydro-9-cis-retinol. In certain cases, the retinol is the all-trans-retinol, 13-cis-retinol, 3,4-didehydro-retinol, or 9-cis-retinol. In certain cases, the retinol is the all-trans-retinol.

A retinyl ester is an ester of retinol. The term “retinol” has been defined above. Retinyl esters suitable for use in the present invention are C1-C30 esters of retinol, such as C2-C20 esters, such as C2, C3, or C16 esters. Examples of retinyl esters include, but are not limited to, retinyl palmitate, retinyl formate, retinyl acetate, retinyl propionate, retinyl butyrate, retinyl valerate, retinyl isovalerate, retinyl hexanoate, retinyl heptanoate, retinyl octanoate, retinyl nonanoate, retinyl decanoate, retinyl undecandate, retinyl taurate, retinyl tridecanoate, retinyl myristate, retinyl pentadecanoate, retinyl heptadeconoate, retinyl stearate, retinyl isostearate, retinyl nonadecanoate, retinyl arachidonate, retinyl behenate, retinyl linoleate, and retinyl oleate.

In some embodiments, the retinyl ester is selected from retinyl palmitate, retinyl acetate and retinyl propionate. In some embodiments, the retinyl ester is retinyl linoleate or retinyl oleate.

The retinoid agent (such as retinol or retinyl ester) is employed in the inventive composition in an amount of from about 0.001% to about 1.5%, such as in an amount of from about 0.01% to about 1%, 0.02% to about 1%, 0.5% to about 1%, 0.10% to about 1%, such as in an amount of from about 0.15% to about 1%.

In some embodiments, the amount of a retinoid agent in the subject composition is at least about 0.01% by weight, at least about 0.02% by weight, at least about 0.03% by weight, at least about 0.04% by weight, at least about 0.5% by weight, at least about 0.6% by weight, at least about 0.7% by weight, at least about 0.8% by weight, at least about 0.9% by weight, at least about 1% by weight, at least about 1.1% by weight, at least about 1.2% by weight, at least about 1.3% by weight, at least about 1.4% by weight, 1.5% by weight, such as at least about 1.4% by weight, at least about 1.3% by weight, at least about 1.2% by weight, at least about 1.0% by weight, at least about 0.9% by weight, at least about 0.8% by weight, at least about 0.7% by weight, at least about 0.6% by weight, at least about 0.5% by weight, or at least about 0.4% by weight. In some embodiments, the subject composition includes about 1.5% by weight or less of a retinoid agent in the non-aqueous solvent solution, such as about 1.2% by weight or less.

In some embodiments, the retinoid agent is tretinoin. In some embodiments, the amount of a tretinoin in the subject composition is at least about 0.01% by weight, at least about 0.02% by weight, at least 0.025% by weight, at least about 0.03% by weight, at least about 0.04% by weight, at least about 0.5% by weight, at least about 0.6% by weight, at least about 0.7% by weight, at least about 0.8% by weight, at least about 0.9% by weight, at least about 1% by weight, least about 1.1% by weight, at least 1.2% by weight, at least 1.3% by weight, at least 1.4% by weight, at least 1.5% by weight, at least 1.6% by weight, at least 1.7% by weight, at least 1.8% by weight, at least 1.9% by weight, or at least 2% by weight. In some embodiments, the amount of tretinoin is about 0.02% by weight. In some embodiments, the amount of tretinoin is about 0.025% by weight.

In some embodiments, the retinoid agent is retinol. In some embodiments, the amount of a tretinoin in the subject composition is at least about 0.01% by weight, at least about 0.02% by weight, at least 0.025% by weight, at least about 0.03% by weight, at least about 0.04% by weight, at least about 0.5% by weight, at least about 0.6% by weight, at least about 0.7% by weight, at least about 0.8% by weight, at least about 0.9% by weight, at least about 1% by weight, least about 1.1% by weight, at least 1.2% by weight, at least 1.3% by weight, at least 1.4% by weight, or at least 1.5% by weight. In some embodiments, the amount of retinol is about 0.08% by weight. In some embodiments, the amount of tretinoin is about 0.09% by weight. In some embodiments, the amount of tretinoin is about 1% by weight. In some embodiments, the amount of tretinoin is about 1.1% by weight.

In some embodiments, the amount of a retinoid agent in the subject composition is at least about 1% by weight, such as at least about 0.9% by weight, at least about 0.8% by weight, at least about 0.7% by weight, at least about 0.6% by weight, at least about 0.5% by weight, at least about 0.4% by weight, at least about 0.3% by weight, at least about 0.2% by weight, at least about 0.15% by weight, or at least about 0.1% by weight. In some embodiments, the subject composition includes about 1% by weight or less of a retinoid agent in the non-aqueous solvent solution, such as about 0.9% by weight or less.

In general, the amounts of retinoid agent in a composition are calculated relative to the solution phase based on the non-aqueous solvent. However, the amounts of retinoid agent and other ingredients relative to the emulsion composition as a whole can readily be calculated by the skilled artisan. It is understood that, in some cases, these concentrate solutions having particular amounts of retinoid agent can be combined with an immiscible ingredient (e.g., an oil component) and an emulsifying agent to produce an emulsion composition (e.g., as described below).

In some embodiments, the subject composition includes about 0.01 to 3% by weight of a retinoid agent (e.g., about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, about 2.6%, about 2.7%, about 2.8%, about 2.9%, or about 3.0%). In some embodiments, the subject composition includes between 0.1-2.5%, about 0.1-2%, or about 0.1-1.5% by weight of a retinoid agent. In certain embodiments, the subject composition includes about 3% by weight of a retinoid agent. In certain embodiments, the subject composition includes about 2% by weight of a retinoid agent. In certain embodiments, the subject composition includes about 1% by weight of a retinoid agent. In certain embodiments, the subject composition includes about 0.01% by weight to 1% by weight of a retinoid agent.

In some embodiments, the subject composition includes about 0.15 to 1.5% by weight of a retinoid agent. In some embodiments, the subject composition includes about 0.2 to 1.4% by weight of a retinoid agent. In certain embodiments, the subject composition includes about 0.3 to 1.3% by weight of a retinoid agent. In certain embodiments, the subject composition includes about 0.4 to 1.2% by weight of a retinoid agent. In certain embodiments, the subject composition includes about 0.5 to 1.1% by weight of a retinoid agent. In certain embodiments, the subject composition includes about 0.5 to 1% by weight of a retinoid agent.

In some embodiments, a second phase (e.g., continuous phase) of the subject composition includes about 0.1 to 1% by weight of a retinoid agent. In some embodiments, the second phase (e.g., continuous phase) of the subject composition includes the second phase (e.g., continuous phase) comprising about 0.1% or more, about 0.2% or more, about 0.3% or more, about 0.4% or more, about 0.5% or more, about 0.6% or more, about 0.7% or more, about 0.8% or more, about 0.9% or more by weight of the retinoid agent. In some embodiments, the second phase (e.g., continuous phase) of the subject composition includes the second phase (e.g., continuous phase) comprising about 1% or less, about 0.9% or less, about 0.8% or less, about 0.7% or less, about 0.6% or less, about 0.5% or less, about 0.4% or less, about 0.3% or less, about 0.2% or less, about 0.1% or less, about 0.08 by weight of the retinoid agent.

### Skin Compatible Solvent

In addition to the urea agent (e.g., as described herein), formulations of the present disclosure contain, as an essential ingredient, at least one non-aqueous skin-compatible solvent. A skin compatible solvent is a solvent that does not cause irritation or sensitization when applied topically to the skin.

In some embodiments, the formulations of the present disclosure comprise a first phase (e.g., dispersed phase) comprising a homogenous solution comprising about 5% or more, about 6% or more, about 7% or more, about 8% or more, about 9% or more, about 10% or more, about 11% or more, about 12% or more, about 13% or more, about 14% or more, about 15% or more, about 16% or more, about 17% or more, about 18% or more, about 19% or more, about 20% or more, about 21% or more, about 22% or more, about 23% or more, about 24% or more, about 25% or more, about 26% or more, about 27% or more, about 28% or more, about 29% or more, about 30% or more, about 31% or more, about 32% or more, about 33% or more, about 34% or more, about 35% or more, about 36% or more, about 37% or more, about 38% or more, about 39% or more, about 40% or more, about 41% or more, about 42% or more, about 43% or more, about 44% or more, about 45% or more, about 46% or more, about 47% or more, about 48% or more, about 49% or more, or about 50% or more by weight of the urea agent; and about 10% or more, about 15% or more, about 20% or more, about 25% or more, about 30% or more, about 35% or more, about 40% or more, about 45% or more, about 50% or more, about 55% or more, about 60% or more, about 65% or more, about 70% or more, about 75% or more, or about 80% or more by weight of a non-aqueous skin-compatible solvent.

In some embodiments, the subject composition includes about 10 to 99% by weight (e.g. about 10% or more, about 15% or more, about 20% or more, about 25% or more, about 30% or more, about 35% or more, about 40% or more, about 45% or more, about 50% or more, about 55% or more, about 60% or more, about 65% or more, about 70% or more, about 75% or more, about 80% or more, about 85% or more, about 90% or more, about 95% or more, about 96% or more, about 97% or more, about 98% or more, or about 99% or more) of a non-aqueous skin compatible solvent. In some embodiments, the subject composition includes about 1 to 30% by weight of an agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10 to 99% polyol. In some embodiments, the subject composition includes about 1 to 30% by weight of an agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10 to 99% polyol and one or more additional skin compatible solvents.

In some embodiments, the non-aqueous skin compatible solvent can be at least 1× the amount of urea, at least 2 times the amount of urea, at least 3 times the amount of urea, at least 4 times the amount of urea, at least 5 times the amount of urea, at least 6 times the amount of urea, or at least 7 times the amount of urea.

In some embodiments, the dispersed phase can be as high as—the dispersed phase can be as high as 70-80%. In other embodiments, the dispersed phase is as low as 20% and can still reach a 5% urea level.

In some embodiments, non-aqueous skin-compatible solvents of interest include polyols, C(1-6) alkanediols, glycol ethers, dimethyl ethers, and combinations thereof.

In some embodiments, the solvent is a skin compatible polyol. A polyol is an organic alcohol solvent having two or more hydroxy groups. In some embodiments, the polyol solvent is a C(3-6)polyol. In some embodiments, the polyol solvent is a polyether polyol. In some embodiments, the polyol solvent is a polyester polyol. Skin compatible polyols of interest include, but are not limited to, glycerol (1,2,3-propanetriol); diglycerol; propylene glycol (1,2-propanediol); dipropylene glycol; 1,3-propanediol; butylene glycol (1,3-butanediol); 1,2-butanediol; pentylene glycol (1,2-pentanediol); 1,5-pentanediol; 1,2-hexanediol; 1,6-hexanediol; 1,2,3-hexanetriol, 1,2,6-hexanetriol; ethoxydiglycol; and dimethyl isosorbide. In some embodiments, the solvent is a glycol ether, a dimethyl ether, or a combination thereof. A preferred skin-compatible solvent is 1,3-propanediol, commercially available from DuPont Tate & Lyle BioProducts LLC under the tradename ZEMEA®. In some embodiments, the solvent is a mixture of 1,3 propanediol and 1,2 hexanediol. In some embodiments, the subject composition includes about 5 to 20% by weight of a urea agent and 1,3-propanediol.

In some embodiments, the subject composition includes about 10 to 99% by weight (e.g. about 10% or more, about 15% or more, about 20% or more, about 25% or more, about 30% or more, about 35% or more, about 40% or more, about 45% or more, about 50% or more, about 55% or more, about 60% or more, about 65% or more, about 70% or more, about 75% or more, about 80% or more, about 85% or more, about 90% or more, about 95% or more, about 96% or more, about 97% or more, about 98% or more, or about 99% or more) of a non-aqueous skin compatible solvent. In some embodiments, the subject composition includes about 1 to 30% by weight of an agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10 to 99% polyol. In some embodiments, the subject composition includes about 1 to 30% by weight of an agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10 to 99% polyol and one or more additional skin compatible solvents.

In some embodiments, the subject composition includes about 10 to 80% by weight (e.g. about 10% or more, about 15% or more, about 20% or more, about 25% or more, about 30% or more, about 35% or more, about 40% or more, about 45% or more, about 50% or more, about 55% or more, about 60% or more, about 65% or more, about 70% or more, about 75% or more, or about 80% or more) of a non-aqueous skin compatible solvent. In some embodiments, the subject composition includes about 1 to 30% by weight of a urea agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10 to 80% polyol. In some embodiments, the subject composition includes between 5-10%, about 10-15%, or about 15-20% by weight of a urea agent, and a polyol. In certain embodiments, the subject composition includes about 5% by weight of a urea agent and 10% to 80% by weight of polyol. In certain embodiments, the subject composition includes about 15% by weight of a urea agent and 10% to 80% by weight of polyol. In certain embodiments, the subject composition includes about 20% by weight of a urea agent and 10% to 80% by weight of polyol.

In some embodiments, the subject composition includes about 7 to 9% by weight urea agent and 10% to 80% by weight of polyol. In certain embodiments, the subject composition includes about 9 to 11% by weight urea agent and 10% to 80% by weight of polyol. In certain embodiments, the subject composition includes about 11 to 13% by weight urea agent and 10% to 80% by weight of polyol. In certain embodiments, the subject composition includes about 13 to 15% by weight urea agent and 10% to 80% by weight of polyol. In certain embodiments, the subject composition includes about 15 to 17% by weight urea agent and 10% to 80% by weight of polyol. In certain embodiments, the subject composition includes about 17 to 19% by weight urea agent and 10% to 80% by weight of polyol. In certain embodiments, the subject composition includes about 19 to 21% by weight urea agent and 10% to 80% by weight of polyol. In certain embodiments, the subject composition includes about 21 to 23% by weight urea agent and 10% to 80% by weight of polyol. In certain embodiments, the subject composition includes about 23 to 25% by weight urea agent and 10% to 80% by weight of polyol.

In some embodiments, the subject composition includes about 1 to 30% by weight of a urea agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and a non-aqueous solvent selected from the group consisting of: C(1-6) alkanediols, glycol ethers, dimethyl ethers. In some embodiments, the subject composition includes between 5-10%, about 10-15%, or about 15-20% by weight of a urea agent, and a non-aqueous solvent selected from the group consisting of: C(1-6) alkanediols, glycol ethers, dimethyl ethers. In certain embodiments, the subject composition includes about 5% by weight of a urea agent and a non-aqueous solvent selected from the group consisting of: C(1-6) alkanediols, glycol ethers, dimethyl ethers. In certain embodiments, the subject composition includes about 15% by weight of a urea agent and a polyol. In certain embodiments, the subject composition includes about 20% by weight of a urea agent and a non-aqueous solvent selected from the group consisting of: C(1-6) alkanediols, glycol ethers, dimethyl ethers.

In some embodiments, the subject composition includes about 5 to 7% by weight of a urea agent and 1,3-propanediol. In some embodiments, the subject composition includes about 7 to 9% by weight urea agent and a non-aqueous solvent selected from the group consisting of: C(1-6) alkanediols, glycol ethers, dimethyl ethers. In certain embodiments, the subject composition includes about 9 to 11% by weight urea agent and a non-aqueous solvent selected from the group consisting of: C(1-6) alkanediols, glycol ethers, dimethyl ethers. In certain embodiments, the subject composition includes about 11 to 13% by weight urea agent and a non-aqueous solvent selected from the group consisting of: C(1-6) alkanediols, glycol ethers, dimethyl ethers. In certain embodiments, the subject composition includes about 13 to 15% by weight urea agent and a non-aqueous solvent selected from the group consisting of: C(1-6) alkanediols, glycol ethers, dimethyl ethers. In certain embodiments, the subject composition includes about 15 to 17% by weight urea agent and a non-aqueous solvent selected from the group consisting of: C(1-6) alkanediols, glycol ethers, dimethyl ethers. In certain embodiments, the subject composition includes about 17 to 19% by weight urea agent and a non-aqueous solvent selected from the group consisting of: C(1-6) alkanediols, glycol ethers, dimethyl ethers. In certain embodiments, the subject composition includes about 19 to 21% by weight urea agent and a non-aqueous solvent selected from the group consisting of: C(1-6) alkanediols, glycol ethers, dimethyl ethers. In certain embodiments, the subject composition includes about 21 to 23% by weight urea agent and 10% to 80% by weight of polyol. In certain embodiments, the subject composition includes about 23 to 25% by weight urea agent and a non-aqueous solvent selected from the group consisting of: C(1-6) alkanediols, glycol ethers, dimethyl ethers.

In some embodiments, the subject composition includes about 1 to 30% by weight of a urea agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10% to 80% by weight of polyether polyol. In some embodiments, the subject composition includes between 5-10%, about 10-15%, or about 15-20% by weight of a urea agent, and 10% to 80% by weight of polyether polyol. In certain embodiments, the subject composition includes about 5% by weight of a urea agent and 10% to 80% by weight of polyether polyol. In certain embodiments, the subject composition includes about 15% by weight of a urea agent and 10% to 80% by weight of polyether polyol. In certain embodiments, the subject composition includes about 20% by weight of a urea agent and 10% to 80% by weight of polyether polyol.

In some embodiments, the subject composition includes about 1 to 30% by weight of a urea agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10% to 80% by weight of polyester polyol. In some embodiments, the subject composition includes between 5-10%, about 10-15%, or about 15-20% by weight of a urea agent, and 10% to 80% by weight of polyester polyol. In certain embodiments, the subject composition includes about 5% by weight of a urea agent and 10% to 80% by weight of polyester polyol. In certain embodiments, the subject composition includes about 15% by weight of a urea agent and 10% to 80% by weight of polyester polyol. In certain embodiments, the subject composition includes about 20% by weight of a urea agent and 10% to 80% by weight of polyester polyol.

In some embodiments, the subject composition includes about 1 to 30% by weight of a urea agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10% to 80% by weight of glycerol (1,2,3-propanetriol). In some embodiments, the subject composition includes between 5-10%, about 10-15%, or about 15-20% by weight of a urea agent, and 10% to 80% by weight of glycerol (1,2,3-propanetriol). In certain embodiments, the subject composition includes about 5% by weight of a urea agent and 10% to 80% by weight of glycerol (1,2,3-propanetriol). In certain embodiments, the subject composition includes about 15% by weight of a urea agent and 10% to 80% by weight of glycerol (1,2,3-propanetriol). In certain embodiments, the subject composition includes about 20% by weight of a urea agent and 10% to 80% by weight of glycerol (1,2,3-propanetriol).

In some embodiments, the subject composition includes about 1 to 30% by weight of a urea agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10% to 80% by weight of diglycerol. In some embodiments, the subject composition includes between 5-10%, about 10-15%, or about 15-20% by weight of a urea agent, and 10% to 80% by weight of diglycerol. In certain embodiments, the subject composition includes about 5% by weight of a urea agent and 10% to 80% by weight of diglycerol. In certain embodiments, the subject composition includes about 15% by weight of a urea agent and 10% to 80% by weight of diglycerol. In certain embodiments, the subject composition includes about 20% by weight of a urea agent and 10% to 80% by weight of diglycerol.

In some embodiments, the subject composition includes about 1 to 30% by weight of a urea agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10% to 80% by weight of propylene glycol (1,2-propanediol). In some embodiments, the subject composition includes between 5-10%, about 10-15%, or about 15-20% by weight of a urea agent, and 10% to 80% by weight of propylene glycol (1,2-propanediol). In certain embodiments, the subject composition includes about 5% by weight of a urea agent and 10% to 80% by weight of propylene glycol (1,2-propanediol). In certain embodiments, the subject composition includes about 15% by weight of a urea agent and 10% to 80% by weight of propylene glycol (1,2-propanediol). In certain embodiments, the subject composition includes about 20% by weight of a urea agent and 10% to 80% by weight of propylene glycol (1,2-propanediol).

In some embodiments, the subject composition includes about 1 to 30% by weight of a urea agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10% to 80% by weight of dipropylene glycol. In some embodiments, the subject composition includes between 5-10%, about 10-15%, or about 15-20% by weight of a urea agent, and 10% to 80% by weight of dipropylene glycol. In certain embodiments, the subject composition includes about 5% by weight of a urea agent and 10% to 80% by weight of dipropylene glycol. In certain embodiments, the subject composition includes about 15% by weight of a urea agent and 10% to 80% by weight of dipropylene glycol. In certain embodiments, the subject composition includes about 20% by weight of a urea agent and 10% to 80% by weight of dipropylene glycol.

In some embodiments, the subject composition includes about 1 to 30% by weight of a urea agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10% to 80% by weight of 1,3-propanediol. In some embodiments, the subject composition includes between 5-10%, about 10-15%, or about 15-20% by weight of a urea agent, and 10% to 80% by weight of 1,3-propanediol. In certain embodiments, the subject composition includes about 5% by weight of a urea agent and 10% to 80% by weight of 1,3-propanediol. In certain embodiments, the subject composition includes about 15% by weight of a urea agent and 10% to 80% by weight of 1,3-propanediol. In certain embodiments, the subject composition includes about 20% by weight of a urea agent and 10% to 80% by weight of 1,3-propanediol. In some embodiments, the subject composition includes about 5 to 7% by weight urea agent and 1,3-propanediol. In some embodiments, the subject composition includes about 7 to 9% by weight urea agent and 1,3-propanediol. In certain embodiments, the subject composition includes about 9 to 11% by weight urea agent and 1,3-propanediol. In certain embodiments, the subject composition includes about 11 to 13% by weight urea agent and 1,3-propanediol. In certain embodiments, the subject composition includes about 13 to 15% by weight urea agent and 1,3-propanediol. In certain embodiments, the subject composition includes about 15 to 17% by weight urea agent and 1,3-propanediol. In certain embodiments, the subject composition includes about 17 to 19% by weight urea agent and 1,3-propanediol. In certain embodiments, the subject composition includes about 19 to 21% by weight urea agent and 1,3-propanediol. In certain embodiments, the subject composition includes about 21 to 23% by weight urea agent and 1,3-propanediol. In certain embodiments, the subject composition includes about 23 to 25% by weight urea agent and 1,3-propanediol.

In some embodiments, the subject composition includes about 1 to 30% by weight of a urea agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10% to 80% by weight of butylene glycol (1,3-butanediol). In some embodiments, the subject composition includes between 5-10%, about 10-15%, or about 15-20% by weight of a urea agent, and 10% to 80% by weight of butylene glycol (1,3-butanediol). In certain embodiments, the subject composition includes about 5% by weight of a urea agent and 10% to 80% by weight of butylene glycol (1,3-butanediol). In certain embodiments, the subject composition includes about 15% by weight of a urea agent and 10% to 80% by weight of butylene glycol (1,3-butanediol). In certain embodiments, the subject composition includes about 20% by weight of a urea agent and 10% to 80% by weight of butylene glycol (1,3-butanediol).

In some embodiments, the subject composition includes about 1 to 30% by weight of a urea agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10% to 80% by weight of 1,2-butanediol. In some embodiments, the subject composition includes between 5-10%, about 10-15%, or about 15-20% by weight of a urea agent, and 10% to 80% by weight of 1,2-butanediol. In certain embodiments, the subject composition includes about 5% by weight of a urea agent and 10% to 80% by weight of 1,2-butanediol. In certain embodiments, the subject composition includes about 15% by weight of a urea agent and 10% to 80% by weight of 1,2-butanediol. In certain embodiments, the subject composition includes about 20% by weight of a urea agent and 10% to 80% by weight of 1,2-butanediol.

In some embodiments, the subject composition includes about 1 to 30% by weight of a urea agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10% to 80% by weight of pentylene glycol (1,2-pentanediol). In some embodiments, the subject composition includes between 5-10%, about 10-15%, or about 15-20% by weight of a urea agent, and 10% to 80% by weight of pentylene glycol (1,2-pentanediol). In certain embodiments, the subject composition includes about 5% by weight of a urea agent and 10% to 80% by weight of pentylene glycol (1,2-pentanediol). In certain embodiments, the subject composition includes about 15% by weight of a urea agent and 10% to 80% by weight of pentylene glycol (1,2-pentanediol). In certain embodiments, the subject composition includes about 20% by weight of a urea agent and 10% to 80% by weight of pentylene glycol (1,2-pentanediol).

In some embodiments, the subject composition includes about 1 to 30% by weight of a urea agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10% to 80% by weight of 1,5-pentanediol. In some embodiments, the subject composition includes between 5-10%, about 10-15%, or about 15-20% by weight of a urea agent, and 10% to 80% by weight of 1,5-pentanediol. In certain embodiments, the subject composition includes about 5% by weight of a urea agent and 10% to 80% by weight of 1,5-pentanediol. In certain embodiments, the subject composition includes about 15% by weight of a urea agent and 10% to 80% by weight of 1,5-pentanediol. In certain embodiments, the subject composition includes about 20% by weight of a urea agent and 10% to 80% by weight of 1,5-pentanediol.

In some embodiments, the subject composition includes about 1 to 30% by weight of a urea agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10% to 80% by weight of 1,2-hexanediol. In some embodiments, the subject composition includes between 5-10%, about 10-15%, or about 15-20% by weight of a urea agent, and 10% to 80% by weight of 1,2-hexanediol. In certain embodiments, the subject composition includes about 5% by weight of a urea agent and 10% to 80% by weight of 1,2-hexanediol. In certain embodiments, the subject composition includes about 15% by weight of a urea agent and 10% to 80% by weight of 1,2-hexanediol. In certain embodiments, the subject composition includes about 20% by weight of a urea agent and 10% to 80% by weight of 1,2-hexanediol.

In some embodiments, the subject composition includes about 1 to 30% by weight of a urea agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10% to 80% by weight of 1,6-hexanediol. In some embodiments, the subject composition includes between 5-10%, about 10-15%, or about 15-20% by weight of a urea agent, and 10% to 80% by weight of 1,6-hexanediol. In certain embodiments, the subject composition includes about 5% by weight of a urea agent and 10% to 80% by weight of 1,6-hexanediol. In certain embodiments, the subject composition includes about 15% by weight of a urea agent and 10% to 80% by weight of 1,6-hexanediol. In certain embodiments, the subject composition includes about 20% by weight of a urea agent and 10% to 80% by weight of 1,6-hexanediol.

In some embodiments, the subject composition includes about 1 to 30% by weight of a urea agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10% to 80% by weight of 1,2,3-hexanetriol. In some embodiments, the subject composition includes between 5-10%, about 10-15%, or about 15-20% by weight of a urea agent, and 10% to 80% by weight of 1,2,3-hexanetriol. In certain embodiments, the subject composition includes about 5% by weight of a urea agent and 10% to 80% by weight of 1,2,3-hexanetriol. In certain embodiments, the subject composition includes about 15% by weight of a urea agent and 10% to 80% by weight of 1,2,3-hexanetriol. In certain embodiments, the subject composition includes about 20% by weight of a urea agent and 10% to 80% by weight of 1,2,3-hexanetriol.

In some embodiments, the subject composition includes about 1 to 30% by weight of a urea agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10% to 80% by weight of 1,2,6-hexanetriol. In some embodiments, the subject composition includes between 5-10%, about 10-15%, or about 15-20% by weight of a urea agent, and 10% to 80% by weight of 1,2,6-hexanetriol. In certain embodiments, the subject composition includes about 5% by weight of a urea agent and 10% to 80% by weight of 1,2,6-hexanetriol. In certain embodiments, the subject composition includes about 15% by weight of a urea agent and 10% to 80% by weight of 1,2,6-hexanetriol. In certain embodiments, the subject composition includes about 20% by weight of a urea agent and 10% to 80% by weight of 1,2,6-hexanetriol.

In some embodiments, the subject composition includes about 1 to 30% by weight of a urea agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10% to 80% by weight of ethoxydiglycol. In some embodiments, the subject composition includes between 5-10%, about 10-15%, or about 15-20% by weight of a urea agent, and 10% to 80% by weight of ethoxydiglycol. In certain embodiments, the subject composition includes about 5% by weight of a urea agent and 10% to 80% by weight of ethoxydiglycol. In certain embodiments, the subject composition includes about 15% by weight of a urea agent and 10% to 80% by weight of ethoxydiglycol. In certain embodiments, the subject composition includes about 20% by weight of a urea agent and 10% to 80% by weight of ethoxydiglycol.

In some embodiments, the subject composition includes about 1 to 30% by weight of a urea agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10% to 80% by weight of dimethyl isosorbide. In some embodiments, the subject composition includes between 5-10%, about 10-15%, or about 15-20% by weight of a urea agent, and 10% to 80% by weight of dimethyl isosorbide. In certain embodiments, the subject composition includes about 5% by weight of a urea agent and 10% to 80% by weight of dimethyl isosorbide. In certain embodiments, the subject composition includes about 15% by weight of a urea agent and 10% to 80% by weight of dimethyl isosorbide. In certain embodiments, the subject composition includes about 20% by weight of a urea agent and 10% to 80% by weight of dimethyl isosorbide.

In some embodiments, the subject composition includes about 1 to 30% by weight of a urea agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10% to 80% by weight of glycol ether, dimethyl ether, or a combination thereof. In some embodiments, the subject composition includes between 5-10%, about 10-15%, or about 15-20% by weight of a urea agent, and 10% to 80% by weight of glycol ether, dimethyl ether, or a combination thereof. In certain embodiments, the subject composition includes about 5% by weight of a urea agent and 10% to 80% by weight of glycol ether, dimethyl ether, or a combination thereof. In certain embodiments, the subject composition includes about 15% by weight of a urea agent and 10% to 80% by weight of glycol ether, dimethyl ether, or a combination thereof. In certain embodiments, the subject composition includes about 20% by weight of a urea agent and 10% to 80% by weight of glycol ether, dimethyl ether, or a combination thereof.

### Emulsifying Agents

It is understood that any of the non-aqueous liquid compositions having particular amounts of a urea agent (e.g., as described herein) can be combined with an immiscible phase (e.g. continuous or silicone/oil phase) or ingredient (e.g., a organosilicone component) to produce an emulsion composition. In some embodiments, the non-aqueous liquid composition that makes up the first phase (e.g., dispersed phase) of an emulsion composition is referred to as a concentrate. The liquid concentrate can be mixed with one or more additional components (e.g., an immiscible oil phase or component, and/or emulsifying agents) to produce the emulsion. A variety of methods and ingredients for preparing emulsions are available and can be used in the subject emulsion compositions.

In some embodiments, an emulsion composition is a gel.

Any convenient oils and lipids can be utilized in the oil component of the subject emulsions. An oil component or oil phase refers to any phase that is immiscible with the non-aqueous liquid composition. In some embodiments, the oil component is silicone-based, e.g., includes a silicone polymer. In some embodiments, the oil component includes a silicone oil or silicone elastomer, such as a polyorganosiloxane. In some embodiments, the silicone polymers have dual characteristics, and can be used as emulsifiers and/or act as the continuous/dispersed phase of the emulsion composition.

Oils and lipids of interest include, but are not limited to, silicone oils, linseed oil, tsubaki oil, macadamia nut oil, corn oil, mink oil, olive oil, avocado oil, sasanqua oil, castor oil, safflower oil, apricot oil, cinnamon oil, jojoba oil, grape oil, sunflower oil, almond oil, rapeseed oil, sesame oil, wheat germ oil, rice germ oil, rice bran oil, cottonseed oil, soybean oil, peanut oil, teaseed oil, evening primrose oil, eggyoke oil, neetsfoot oil, liver oil, triglycerine, glycerine trioctanate, pentaerythritol tetraoctanate, glycerine triisopalmitate, cholesterol, free fatty acids, and combinations thereof.

Any convenient emulsifying agents or emulsifiers can be utilized in the preparation of the subject emulsions to stabilize the composition and prevent separation of the oil component from the solvent solution (e.g., the non-aqueous liquid composition of the first phase (e.g., dispersed phase) solution).

In some embodiments, the formulations of the present disclosure contain about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, or about 25% by weight of an emulsifying agent. In some embodiments, the formulations of the present disclosure contain an emulsifying agent ranging from about 4% to about 6%, about 6% to about 8%, about 8% to about 10%, about 10% to about 12% about 14%, about 14% to about 16%, about 16% to about 18%, about 18% to about 20%, about 20% to about 22%, about 22% to about 24%, about 24% to about 26%, about 26% to about 28%, or about 28% to about 30% by weight.

In some embodiments, the emulsifying agent is a silicone-compatible agent.

In some embodiments, emulsifying agents include, but are not limited to, polysorbates, laureth-4, potassium cetyl sulfate, and silicone and silicone-elastomer-based emulsifiers and emulsifying blends. In some embodiments, a surfactant such as a monoglyceride, sorbitan fatty acid ester, or polyglycerine fatty acid ester, polyoxyethylene hardened castor oil, polyoxyethylene fatty acid ether, or combinations thereof, is added thereto in a small amount, and the stability is further improved. In some embodiments, the emulsifying agent is added to the formulation in an effective amount to improve the stability of the formulation.

In some embodiments, the emulsifying agents include, but are not limited to: sorbitan laurate, sorbitan palmitate, sorbitan sesquiisostearate, sorbitan sesquioleate, sorbitan sesquistearate, sorbitan stearate, sorbitan oleate, sorbitan monoisostearate, sorbitan trisostearate, sorbitan trioleate, sorbitan tristearate; glyceryl behenate, glyceryl caprate, glyceryl caprylate, glyceryl caprylate/caprate, glyceryl cocoate, glyceryl erucate, glyceryl hydroxystearate, glyceryl isostearate, glyceryl lanolate, glyceryl laurate, glyceryl linoleate, glyceryl myristate, glyceryl oleate, glyceryl palmitate lactate, glyceryl sesquioleate, glyceryl stearate, glyceryl stearate citrate, glyceryl stearate lactate; polyglyceryl-4 isostearate, polyglyceryl-3 oleate, polyglyceryl-2 sesquioleate, triglyceryl diisostearate, diglyceryl monooleate, tetraglyceryl monooleate, glycol distearate, glycol hydroxystearate, glycol oleate, glycol ricinoleate, glycol stearate, propylene glycol isostearate, propylene glycol hydroxystearate, propylene glycol laurate, propylene glycol myristate, propylene glycol oleate, propylene glycol ricinoleate, propylene glycol stearate; sucrose cocoate, sucrose laurate; Methyl Glucose Sesquistearate, Methyl Glucose Dioleate; PEG-20 Methyl Glucose Sesquistearate; or mixtures thereof.

### Silicone Agents

In some embodiments, the emulsifying agent of the formulations of the present disclosure contains one or more silicone emulsifiers (e.g. silicone agents). In some embodiments, a first phase (e.g., dispersed phase) solution containing a urea agent and a non-aqueous solvent (e.g. the non-aqueous liquid composition) is mixed with a second phase (e.g., continuous phase) solution containing one or more silicone emulsifying agents to form an emulsion formulation. In some embodiments, the emulsion prevents degradation of the urea agent. In some embodiments, the emulsion prevents precipitation of the urea agent. In some embodiments, the emulsion prevents oxygen or air from degrading the urea agent within the emulsion. In some embodiments, the emulsion prevents moisture from degrading the urea agent within the emulsion. In some embodiments, the emulsion prevents absorption of water from degrading the urea agent within the emulsion.

In some embodiments, the emulsion increases the shelf life of the emulsion containing the urea agent. In some embodiments, the shelf life of the emulsion ranges from 6 weeks to 8 weeks, 2 months 4 months, 4 months to 6 months, 6 months to 1 year, 1 to 1.5 years, 1.5 to 2 years, 2 to 2.5 years, 2.5 to 3 years, 3 to 3.5 years, or 3.5 to 4 years. In some embodiments, the shelf life is about 1 month or more, about 2 months or more, about 3 months or more, about 4 months or more, about 5 months or more, about 6 months or more, about 1 year or more, about 1.5 years or more, about 2 years or more, or about 2.5 years or more.

Silicone agents of interest include, but are not limited to, dimethicone, adimethicone, cyclopentasiloxane, dimethicone/PEG-10/15 crosspolymer, lauryl PEG-9 polydimethylsiloxyethyl dimethicone, PEG-3 dimethicone, PEG-10 dimethicone, PEG-9 methyl ether dimethicone, polyglyceryl-3 polydimethylsiloxyethyl dimethicone, PEG/PPG-18/18 dimethicone, PEG-15/lauryl dimethicone crosspolymer, PEG-15/lauryl polydimethylsiloxyethyl dimethicone crosspolymer, dimethicone/polyglycerin-3 crosspolymer, and dimethicone/vinyl eimethicone crosspolymer.

In some embodiments, the formulations of the present disclosure contain about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30% by weight of one or more silicone agents. In some embodiments, the formulations of the present disclosure contain an amount of one or more silicone agents ranging from about 1 to 5%, 5 to 10%, 10 to 15%, 15 to 20%, 20 to 25%, or 25 to 30% by weight.

In some embodiments, the composition of the present disclosure includes second phase (e.g., continuous phase or silicone/oil phase) comprising a silicone agent. In some embodiments, the second phase (e.g., continuous phase) contains about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30% by weight of one or more silicone agents. In some embodiments, the second phase (e.g., continuous phase) contains an amount of one or more silicone agents ranging from about 1 to 10%, 10 to 20%, 20 to 30%, 30 to 40%, 40 to 50%, 50 to 60%, 60 to 70%, 70 to 80%, 80 to 90%, or 90 to 100% by weight. In some embodiments, the second phase (e.g., continuous phase) contains an amount of one or more silicone agents ranging from about 1 to 5%, 5 to 10%, 10 to 15%, 15 to 20%, 20 to 25%, 25 to 30%, 30 to 35%, 35 to 40%, 40 to 45%, 45 to 50%, 50 to 55%, 55 to 60%, 60 to 65%, 65 to 70%, 70 to 75%, 75 to 80%, 80 to 85%, 85 to 90%, 90 to 95%, or 95 to 100% by weight.

In some embodiments, the second phase (e.g., continuous phase) comprises, in addition to one or more silicone agents, a non-silicone oil agent. In some embodiments, the second phase (e.g., continuous phase) comprises, in addition to one or more silicone agents, 0.5 to 1%, 1 to 1.5%, 1.5 to 2%, 2 to 2.5%, 2.5 to 3%, 3 to 3.5%, 3.5 to 4%, 4 to 4.5%, 4.5 to 5%, 5 to 5.5%, 6 to 6.5%, 6.5 to 7%, 7 to 7.5%, 8 to 8.5%, 8.5% to 9%, 9 to 9.5%, or 9.5 to 10% by weight of a non-silicone oil agent. In some embodiments, the second phase (e.g., continuous phase) contains an amount of one or more silicone agents ranging from about 1 to 5%, 5 to 10%, 10 to 15%, 15 to 20%, 20 to 25%, 25 to 30%, 30 to 35%, 35 to 40%, 40 to 45%, 45 to 50%, 50 to 55%, 55 to 60%, 60 to 65%, 65 to 70%, 70 to 75%, 75 to 80%, 80 to 85%, 85 to 90%, 90 to 95%, or 95 to 100% by weight; and 0.5 to 1%, 1 to 1.5%, 1.5 to 2%, 2 to 2.5%, 2.5 to 3%, 3 to 3.5%, 3.5 to 4%, 4 to 4.5%, 4.5 to 5%, 5 to 5.5%, 6 to 6.5%, 6.5 to 7%, 7 to 7.5%, 8 to 8.5%, 8.5% to 9%, 9 to 9.5%, or 9.5 to 10% by weight of a non-silicone oil agent.

In some embodiments, the silicone agent is dimethicone. In some embodiments, the silicone agent contains dimethicone in combination with a dimethicone/PEG-10/15 crosspolymer. In some embodiments, the silicone agent contains dimethicone in combination with a dimethicone/PEG-10/15 crosspolymer and lauryl PEG-9 polydimethylsiloxyethyl

dimethicone. In some embodiments, the silicone agent contains about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30% dimethicone alone or in combination with a dimethicone/PEG-10/15 crosspolymer and/or lauryl PEG-9 polydimethylsiloxyethyl. In some embodiments, the silicone agent contains about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30% dimethicone; about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30% dimethicone/PEG-10/15 crosspolymer; and/or about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30% lauryl PEG-9 polydimethylsiloxyethyl.

In some embodiments, the subject composition includes a ratio of the first phase (e.g., dispersed phase) to the second phase (e.g., continuous phase). In certain embodiments, the ratio of the first phase (e.g., dispersed phase) to the second phase (e.g., continuous phase) ranges from 1 to 5, such as a ratio of 1.0 (i.e., 1:1), 1.25, 1.50, 1.75, 2.0 (i.e. 2:1), 2.25, 2.50, 2.75, 3.0, 3.25, 3.50, 3.75, 4.0, 4.25, 4.50, 4.75, 5. In certain embodiments, the ratio of the first phase (e.g., dispersed phase) to the second phase (e.g., continuous phase) ranges from 1 to 20, such as a ratio of 1 (i.e., 1:1), 2, 3, 4, 5, 6, 7, 8, 9, 10, 11, 12, 13, 14, 15, 16, 17, 18, 19, or 20. In certain embodiments, the ratio of the first phase (e.g., dispersed phase) to the second phase (e.g., continuous phase) is 1.8 to 2.2, such as a ratio of 2. In certain embodiments, the ratio of the first phase (e.g., dispersed phase) to the second phase (e.g., continuous phase) is 1.0 to 1.3, such as a ratio of 1.25 or a ratio of 1.0. In some embodiments, the ratio of the first phase (e.g., dispersed phase) to the second phase (e.g., continuous phase) is up to 19 (e.g., 19:1). In some embodiments, the ratio of the first phase (e.g., dispersed phase) to the second phase (e.g., continuous phase) ranges from 1 to 19. In some embodiments, the ratio of the first phase (e.g., dispersed phase) to the second phase (e.g., continuous phase) is 9 (i.e., 9:1). In some embodiments, the ratio of the first phase (e.g., dispersed phase) to the second phase (e.g., continuous phase) is 19 (i.e., 19:1).

In some embodiments, the first phase is an dispersed phase and the second phase is an continuous phase. In some embodiments, droplets of the dispersed phase (e.g., solvent phase) are contained within the continuous phase (e.g., silicone phase).

### Additional Components

A formulation may contain one or more (optional) additional ingredients. Any convenient ingredient known to the skilled artisan to provide cosmetic/aesthetic benefits can be utilized in the subject formulations. Such cosmetic/aesthetic benefits include, but are not limited to, reducing the appearance of fine lines/wrinkles, improving skin barrier function (by reducing the rate/extent of trans-epidermal water loss), making the skin feel smoother/more supple/softer, creating the appearance of more even skin tone (reducing dyschromia) and/or “glow”/radiance (also described in the art as “brightness”).

In some embodiments, the composition further includes one or more optional additional components (e.g., as described herein). In some embodiments, one or more additional components is an antioxidant. In some cases, the antioxidant is selected from ascorbic acid and azelaic acid. In some embodiments, the one or more optional additional components are selected from physiologic lipids, ascorbic acid, retinol, tocopherols, tocotrienols (e.g., alpha, beta, delta and gamma tocopherols or alpha, beta, delta and gamma tocotrienols), ferulic acid, azelaic acid, hydroxy acids (e.g., salicylic acid), panthenol, Pinus pinaster bark extract, emulsifying agent, hyaluronic acid complex, madecassoside, madecassoside asiaticoside, acetyl zingerone, bakuchiol, and bis-ethylhexyl hydroxydimethoxy benzylmalonate, zinc oxide, and titanium dioxide.

Each optional additional component (e.g., as described herein) may be present in an amount of 20% or less by weight of the composition, such as 19% or less, 18% or less, 17% or less, 16% or less, 15% or less, 14% or less, 13% or less, 12% or less, 11% or less, 10% or less, 9% or less, 8% or less, 7% or less, 6% or less, 5% or less, 4% or less, 3% or less, 2% or less, 1% or less, 0.5% or less, 0.5% or less, 0.4% or less, 0.3% or less, 0.2% or less, or 0.1% or less by weight. In some embodiments the total amount of the one or more optional additional components (e.g., as described herein) in the composition 10% or less by weight, such as 9% or less, 8% or less, 7% or less, 6% or less, 5% or less, 4% or less, 3% or less, 2% or less, 1% or less, 0.5% or less, 0.4% or less, 0.3% or less, 0.2% or less, or 0.1% or less by weight.

In some embodiments, the composition further includes 10% or less by weight in total of one or more optional additional components selected from an antioxidant, a skin lightening agent, and a moisturizing agent.

In some embodiments, the first phase (e.g., dispersed phase) solution of the subject composition includes one or more (optional) additional ingredients described herein. Each optional additional component (e.g., as described herein) may be present in the first phase (e.g., dispersed phase) in an amount of 20% or less by weight of the first phase (e.g., dispersed phase), such as 19% or less, 18% or less, 17% or less, 16% or less, 15% or less, 14% or less, 13% or less, 12% or less, 11% or less, 10% or less, 9% or less, 8% or less, 7% or less, 6% or less, 5% or less, 4% or less, 3% or less, 2% or less, 1% or less, 0.5% or less, 0.5% or less, 0.4% or less, 0.3% or less, 0.2% or less, or 0.1% or less by weight. In some embodiments the amount of the one or more optional additional components (e.g., as described herein) dissolved in the first phase (e.g., dispersed phase) is 10% or less by weight, such as 9% or less, 8% or less, 7% or less, 6% or less, 5% or less, 4% or less, 3% or less, 2% or less, 1% or less, 0.5% or less, 0.4% or less, 0.3% or less, 0.2% or less, or 0.1% or less by weight.

In some embodiments, the second phase (e.g., continuous phase) solution of the subject composition includes one or more (optional) additional ingredients described herein. Each optional additional component (e.g., as described herein) may be present in the first phase (e.g., dispersed phase) in an amount of 20% or less by weight of the first phase (e.g., dispersed phase), such as 19% or less, 18% or less, 17% or less, 16% or less, 15% or less, 14% or less, 13% or less, 12% or less, 11% or less, 10% or less, 9% or less, 8% or less, 7% or less, 6% or less, 5% or less, 4% or less, 3% or less, 2% or less, 1% or less, 0.5% or less, 0.5% or less, 0.4% or less, 0.3% or less, 0.2% or less, or 0.1% or less by weight. In some embodiments the amount of the one or more optional additional components (e.g., as described herein) dissolved in the first phase (e.g., dispersed phase) is 10% or less by weight, such as 9% or less, 8% or less, 7% or less, 6% or less, 5% or less, 4% or less, 3% or less, 2% or less, 1% or less, 0.5% or less, 0.4% or less, 0.3% or less, 0.2% or less, or 0.1% or less by weight.

### Physiologic Lipids

In some embodiments, the composition includes an optional additional component that is a physiologic lipid. In some embodiments, the physiologic lipid is a ceramide, cholesterol, cholesterol ester, a free fatty acid, and combinations thereof.

In some embodiments, the physiologic lipid agent is present in the composition in an amount of 8% or less by weight, such as 7% or less, 6% or less, or 5% or 4% or less, or 3% or less, or 2% or less, 1% or less, 0.5% or less, 0.4% or less, 0.3% or less, 0.2% or less, or 0.1% or less by weight.

### Ascorbic Acid

The terms “ascorbic acid”, “L-ascorbic acid” and “vitamin C” are used interchangeably herein, and refer to the naturally occurring vitamin of CAS Registry Number: 50-81-7. Any convenient form of ascorbic acid can be utilized in the subject formulations. In some embodiments, the ascorbic acid used of the present disclosure is a powder.

In certain embodiments, the ascorbic acid material used in preparing the subject compositions is composed of granular particles. Such a particulate powder has a particle size (e.g., mean particle size) of less than about 25 microns, such as less than about 20 microns, and less than about 12.5 microns, e.g., as measured by a Hagman gauge. In some embodiments, all of the ascorbic acid powder used in preparing the subject compositions is capable of passage through a No. 100 U.S. Standard Sieve, a standard testing procedure used by the US Pharmacopoeia. In some embodiments, 80% or more (such as 90% or more, or 100%) of ascorbic acid powder used in preparing the subject composition is capable of passage through a No. 325 U.S. Standard Sieve. For example, one powder meeting the above criterion is Ascorbic Acid Ultra-Fine Powder from DSM Nutritional Products LLC, Parsippany, NJ. Previously, this product was available as Product Code No. 6045653 from Roche Vitamins and Fine Chemicals.

In some embodiments, the amount of ascorbic acid in the subject composition is from 5 to 30% by weight, such as 5 to 28%, 5 to 25%, 5 to 20%, or 5 to 15% by weight.

In some embodiments, the amount of ascorbic acid in the subject composition is at least about 5% by weight, such as at least about 10% by weight, at least about 12% by weight, at least about 15% by weight, at least about 20% by weight.

In some embodiments, the amount of ascorbic acid in the subject composition is at least about 5% by weight, such as at least about 10% by weight, at least about 12% by weight, at least about 15% by weight, at least about 20% by weight, or at least about 25% by weight. In some embodiments, the subject composition includes about 28% by weight or less of ascorbic acid in the non-aqueous solvent solution, such as about 25% by weight or less. In certain embodiments, the non-aqueous solvent is a polyol. In certain embodiments, the non-aqueous solvent is 1,3-propanediol. In particular embodiments, the amount of ascorbic acid in the subject composition is between about 5% by weight and about 22% by weight, about 10% by weight and about 20% by weight, or between about 12% by weight and about 28% by weight, such as between 5% by weight and about 15% by weight, such as between about 15% by weight and about 28% by weight, or between about 20% by weight and about 28% by weight. In some embodiments, the amount of ascorbic acid in the subject composition is about 5%, about 10%, about 15%, about 20%, or about 25% by weight.

In some embodiments, the amount of ascorbic acid in the subject composition is about 5%, about 6%, about 7%, about 8%, about 9%, about 10%, about 11%, about 12%, about 13%, about 14%, about 16%, about 17%, about 18%, about 19%, about 20%, about 21%, about 22%, about 23%, about 24%, about 25%, about 26%, about 27%, about 28%, about 29%, or about 30% by weight.

In particular embodiments, the amount of ascorbic acid in the subject composition is between about 25% by weight and about 28% by weight (e.g., about 25%, about 26%, about 27% or about 28%) where the ratio of ascorbic acid to urea agent (% wt ratio) is 1.0 to 1.3, such as a ratio of 1.25 (i.e., 1.25:1) or a ratio of 1.0 (i.e., 1:1).

### Azelaic Acid

Azelaic acid, or nonanedoic acid is a naturally occurring dicarboxylic acid of the chemical formula HO₂C(CH₂)₇CO₂H. Azelaic acid is produced by the yeast species Malassezia furfur and found in whole grain cereals, rye, barley and animal products. Azelaic acid is known to possess antibacterial, keratolytic, comedolytic, and antioxidant activity. Azelaic acid is bactericidal against Propionibacterium acnes and Staphylococcus epidermidis due to its inhibitory effect on the synthesis of microbial cellular proteins. Azelaic acid exerts its keratolytic and comedolytic effects by reducing the thickness of the stratum corneum and decreasing the number of keratohyalin granules by reducing the amount and distribution of filaggrin in epidermal layers. Azelaic acid is also known to possess a direct anti-inflammatory effect due to its scavenger activity of free oxygen radical. Azelaic acid has been used topically to reduce inflammation associated with acne and rosacea.

In some embodiments, the amount of azelaic acid in the subject composition is from 5 to 30% by weight, such as 5 to 28%, 5 to 25%, 5 to 20%, or 5 to 15% by weight.

In some embodiments, the amount of azelaic acid in the subject composition is at least about 5% by weight, such as at least about 10% by weight, at least about 12% by weight, at least about 15% by weight, at least about 20% by weight.

In some embodiments, the amount of azelaic acid in the subject composition is at least about 5% by weight, such as at least about 10% by weight, at least about 12% by weight, at least about 15% by weight, at least about 20% by weight, or at least about 25% by weight. In some embodiments, the subject composition includes about 28% by weight or less of azelaic acid in the non-aqueous solvent solution, such as about 25% by weight or less. In certain embodiments, the non-aqueous solvent is a polyol. In certain embodiments, the non-aqueous solvent is 1,3-propanediol. In particular embodiments, the amount of azelaic acid in the subject composition is between about 5% by weight and about 22% by weight, about 10% by weight and about 20% by weight, or between about 12% by weight and about 28% by weight, such as between 5% by weight and about 15% by weight, such as between about 15% by weight and about 28% by weight, or between about 20% by weight and about 28% by weight. In some embodiments, the amount of azelaic acid in the subject composition is about 5%, about 10%, about 15%, about 20%, or about 25% by weight.

In some embodiments, the amount of azelaic acid in the subject composition is about 5%, about 6%, about 7%, about 8%, about 9%, about 10%, about 11%, about 12%, about 13%, about 14%, about 16%, about 17%, about 18%, about 19%, about 20%, about 21%, about 22%, about 23%, about 24%, about 25%, about 26%, about 27%, about 28%, about 29%, or about 30% by weight.

In particular embodiments, the amount of azelaic acid in the subject composition is between about 25% by weight and about 28% by weight (e.g., about 25%, about 26%, about 27% or about 28%) where the ratio of azelaic acid to urea agent (% wt ratio) is 1.0 to 1.3, such as a ratio of 1.25 (i.e., 1.25:1) or a ratio of 1.0 (i.e., 1:1).

### Tocopherol or Tocotrienol Agent

In some embodiments, the composition further includes optional additional component that is a tocopherol or tocotrienol agent. In some embodiments, the tocopherol or tocotrienol agent is a form of Vitamin E selected from alpha, beta, delta and gamma tocopherols and alpha, beta, delta and gamma tocotrienols, and combinations thereof. In some embodiments, the tocopherol or tocotrienol is alpha-tocopherol.

In some embodiments, the tocopherol or tocotrienol agent is present in the composition in an amount of 2% or less by weight, such as 1.5% or less, 1% or less, 0.5% or less, 0.4% or less, 0.3% or less, 0.2% or less, or 0.1% or less by weight.

In some embodiments of any one of the formulations described herein, the formulation excludes tocopherol or tocotrienol agents, e.g., or precursors thereof having vitamin E activity. In certain embodiments of any one of the formulations described herein, the formulation excludes vitamin E acetate.

### Additional Antioxidants

In certain embodiments, the formulation contains a secondary antioxidant (i.e., in addition to Vitamin C, azelaic acid or the optional additive tocopherol or tocotrienol agent).

Preferred secondary antioxidants include cinnamic acid derivatives (e.g., ferulic acid, caffeic acid, or coumaric acid), terpenoid antioxidants, and benzoic acid derivatives (e.g., p-hydroxy benzoic acid, gallic acid, or protocatechuic acid). Pinus Pinaster Bark/Bud Extract (available under the tradename Pycnogenol® from DKSH North America, Inc., or from Res Pharma Industriale under the tradename Pantrofina® Skin360) contains these cinnamic acid derivatives and benzoic acid derivatives, and is, therefore, a preferred secondary antioxidant.

In some embodiments, the secondary antioxidant is zingerone or acetyl zingerone. In some embodiments, the secondary antioxidant is bakuchiol (10309-37-2) a natural terpenoid antioxidant. In some embodiments, the secondary antioxidant is bis-ethylhexyl hydroxydimethoxy benzylmalonate (HDBM).

In some embodiments, the secondary antioxidant, when included, is present in an amount in the range of 0.1 to 3%, more 0.1 to 2% by weight of the composition, such as 0.1 to 1% by weight, 0.1 to 0.5% by weight, e.g., about 0.2%, about 0.3%, about 0.4% or about 0.5% by weight. In some embodiments, the secondary antioxidant is acetyl zingerone.

In some embodiments, the secondary antioxidant, when included, is present in an amount of 5% or less, 4% or less, or 3% or less, or 2% or less, 1% or less, 0.5% or less, 0.4% or less, 0.3% or less, 0.2% or less, or 0.1% or less by weight.

### Skin Lightening Agents

In certain embodiments, the formulation contains a secondary skin lightening agent (e.g., as defined herein) (i.e., in addition to the retinoid agent and Vitamin C). Skin lightening agents which may be included in compositions of the present disclosure include, but are not limited to: hydroquinone and its derivatives, including, for example, its monomethyl and monobenzyl ethers; licorice root (Glycyrrhiza glabra) extract; azelaic acid; kojic acid; arbutin; alpha hydroxy acids, in particular citric acid, lactic acid, and glycolic acid; ellagic acid; gluconic acid; gentisic acid (2,5-dihydrobenzoic acid); 4-hydroxy benzoic acid; salts and esters of the above-mentioned acids, including ammonium lactate and sodium lactate; N-acetyl glucosamine; aloesin, a hydroxymethyl chromone isolated from aloe vera; Vitamin B3 compound or its derivative—niacin, nicotinic acid, niacinamide. Epigallocatechin 3-O-gallate (EGCG), and other catechin constituents of tea extracts, in particular green tea; extract of soybean oil (Glycine soja), including isoflavones; hydroxystilbene; butyl hydroxy anisole; and butyl hydroxy toluene may also be utilized as a skin lightening agent. In some embodiments, the additional skin lightening agent is azelaic acid or arbutin.

The skin lightening agent, when included, is present in an amount in the range of 0.1 to 10%, 0.2 to 5% by weight of the composition, such as 0.2 to 4% by weight, 0.2 to 3% by weight, or 0.2 to 2% by weight. In certain embodiments, the secondary skin lightening agent is soluble and may be added directly to the high Vitamin C (>15%) concentrate of the present invention. The secondary skin lightening agent may also be encapsulated using techniques known to the person having ordinary skill in the art.

### Hydroxy Acids

In some embodiments, formulation contains a hydroxy acid, e.g., a small molecule compound including a carboxylic acid and a hydroxy group. The acid may be an alkyl carboxylic acid or a benzoic acid. The hydroxy group can be a phenol or an alkyl alcohol. In certain embodiments, the hydroxy acid is an alpha-hydroxy carboxylic acid. In certain embodiments the hydroxy acid contains 2-12 carbon atoms, such as 2-6 or 2-4 carbons. Hydroxy acids of interest include, but are not limited to, glycolic acid, lactic acid, mandelic acid, salicylic acid, capryloyl salicylic acid, salicyloyl phytosphingosine, gluconolactone, lactobionic acid, maltobionic acid, and combinations thereof.

### Anti-Inflammatory

In some embodiments, formulation contains an anti-inflammatory agent as an additional ingredient. In some embodiments, the anti-inflammatory agent is madecassoside, madecassoside asiaticoside, or madecassic acid. The anti-inflammatory agent, when included, is present in an amount in the range of 0.1 to 2%, 0.1 to 1% by weight of the composition, such as 0.1 to 0.5% by weight, or 0.1 to 0.2% by weight. In some embodiments, madecassoside or madecassoside asiaticoside is included in an amount in the range of 0.1 to 0.5%, such as about 0.1% or about 0.2% by weight.

### Exemplary Topical Formulations

In some embodiments, the topical composition includes: a) 1% to 30% by weight urea; b) 0.01% to 1.5% by weight retinoid agent; and c) 10% to 80% by weight of a non-aqueous skin-compatible solvent selected from polyol, C(1-6) alkanediol, glycol ether, dimethyl ether, or a combination thereof, d) 4% to 24% of a silicone agent selected from a linear silicone, branched silicone, silicone crosspolymer, or a combination thereof.

In some embodiments, the topical composition includes: about 10% by weight urea agent; about 0.2% by weight retinoid agent; about 46% by weight a solvent that includes 1,3-propanediol and/or 1,2-hexanediol; and one or more optional additional components; 19% by weight silicone agent; and one or more optional additional components. In certain embodiments, one or more additional component is an antioxidant. In certain embodiments, the topical composition includes 5 to 22% by weight of an antioxidant. In certain embodiments, one or more antioxidants is ascorbic acid. In certain embodiments, one or more antioxidants is azelaic acid. In certain embodiments, one or more optional additional component is a tocopherol or tocotrienol (e.g., as described herein). In certain embodiments, one or more optional additional component is ferulic acid. In certain embodiments, one or more optional additional component is bis-ethylhexyl hydroxydimethoxy benzylmalonate.

In some embodiments, the topical composition includes: about 5% by weight urea agent; about 0.2% by weight retinoid agent; about 45.3% by weight a solvent that includes 1,3-propanediol and/or 1,2-hexanediol; and one or more optional additional components; 24% by weight silicone agent; and one or more optional additional components. In certain embodiments, one or more additional component is an antioxidant. In certain embodiments, the topical composition includes 5 to 15% by weight of an antioxidant. In certain embodiments, the one or more optional additional component is ascorbic acid. In certain embodiments, one or more optional additional component is azelaic acid. In certain embodiments, one or more optional additional component is a tocopherol or tocotrienol (e.g., as described herein). In certain embodiments, one or more optional additional component is madecassoside. In certain embodiments, one or more optional additional components is madecassoside asiaticoside. In certain embodiments, one or more optional additional component is ferulic acid. In certain embodiments, one or more optional additional component is glycyrrhetinic acid. In certain embodiments, one or more optional additional component is Pinus pinaster bark extract. In certain embodiments, one or more optional additional component is cholesterol ester. In certain embodiments, one or more optional additional component is bakuchiol.

### Containers

Any containers suitable for storing and/or dispensing the subject formulations can be adapted for use. The container can provide a sealed environment for containing the composition, and separation from the atmosphere. The container can prevent during storage undesirable degradation, e.g., from absorption of light and/or moisture from the atmosphere or surrounding environment. Provided are ready-to-use topical preparations of a urea agent in a multi-use container which is pre-filled with a storage stable topical composition (e.g., as described herein).

The container will include a delivery system of urea that combines both solubilization (e.g., for better delivery to skin) with a “time released” vehicle that will minimize potential irritation. For example, a solubilized urea-containing polyol phase is dispersed into small droplets inside of a continuous silicone/oil outer phase that can deliver the urea efficiently but not all at once.

Additional packaging for the container can be included. In some cases, the packaging provides a further barrier that prevents absorption of light and/or moisture from the atmosphere or surrounding environment.

In certain embodiments, the container is a glass container, such as a glass jar. In certain other embodiments, the container is a plastic container. In certain embodiments, the container includes a dispensing means, such as an airless pump.

### Kits

Another aspect of the present disclosure provides a kit comprising the composition of the present disclosure, and optionally instructions for using the composition. In some embodiments, the kit comprises instructions for using the kit. In some embodiments, the instructions for using the kit comprises: gently applying to the skin (e.g., face) % to 1 pump of the any one of formulations 1-15 of the present disclosure, wherein the composition is applied in the morning, in the evening, once daily, twice daily, or as needed. In some embodiments, the composition is applied daily. In some embodiments, the composition is applied twice daily. In some embodiments, the composition is applied as needed.

### Physical Properties

### Viscosity

Aspects of the present disclosure includes emulsion formulations with a viscosity ranging from 45,000 cps to 100,000 cps. The viscosity of the formulations of the present disclosure may change over time.

In some embodiments, the viscosity ranges from 45,000 cps to 100,000 cps cps. In some embodiments, the viscosity ranges from 50,000 cps to 100,000 cps. In some embodiments, the viscosity ranges from 51,000 cps to 100,000 eps. In some embodiments, the viscosity ranges from 40,000 cps to 100,000 cps. In some embodiments, the viscosity ranges from 45,000 cps to 100,000 cps cps after storage for at least 3 years at room temperature (25° C.). In certain embodiments, the viscosity ranges from 45,000 cps to 100,000 cps at the time of manufacture. In some embodiments, the viscosity ranges from 45,000 cps to 100,000 cps after storage for at least 3 years at room temperature (25° C.). In some embodiments, the viscosity ranges from 45,000 cps to 100,000 cps after storage for at least 1 year at room temperature (25° C.). In some embodiments, the viscosity ranges from 45,000 cps to 100,000 cps after storage for at least 2 years at room temperature (25° C.). In some embodiments, the viscosity ranges from 45,000 cps to 100,000 cps after storage for at least 6 months at room temperature (25° C.). In some embodiments, the viscosity ranges from 45,000 cps to 100,000 cps after storage for at least 1 year at 5° C. In some embodiments, the viscosity ranges from 45,000 cps to 100,000 cps after storage for at least 2 years at 5° C. In some embodiments, the viscosity ranges from 45,000 cps to 100,000 cps after storage for at least 3 years at 5° C. In some embodiments, the viscosity ranges from 45,000 cps to 100,000 cps after storage for at least 6 months at 5° C. In some embodiments, the viscosity ranges from 45,000 cps to 100,000 cps after storage for at least 1 year at 20° C. In some embodiments, the viscosity ranges from 45,000 cps to 100,000 cps after storage for at least 2 years at 20° C. In some embodiments, the viscosity ranges from 45,000 cps to 100,000 cps after storage for at least 3 years at 20° C. In some embodiments, the viscosity ranges from 45,000 cps to 100,000 cps after storage for at least 6 months at 20° C. In some embodiments, the viscosity ranges from 45,000 cps to 100,000 cps after storage for at least 1 year at 40° C. In some embodiments, the viscosity ranges from 45,000 cps to 100,000 cps after storage for at least 2 years at 40° C. In some embodiments, the viscosity ranges from 45,000 cps to 100,000 cps after storage for at least 3 years at 40° C. In some embodiments, the viscosity ranges from 45,000 cps to 100,000 cps after storage for at least 6 months at 40° C. In some embodiments, the viscosity ranges from 45,000 cps to 100,000 cps after storage for at least 1 year at 45° C. In some embodiments, the viscosity ranges from 45,000 cps to 100,000 cps after storage for at least 2 years at 45° C. In some embodiments, the viscosity ranges from 45,000 cps to 100,000 cps after storage for at least 3 years at 45° C. In some embodiments, the viscosity ranges from 45,000 cps to 100,000 cps after storage for at least 6 months at 45° C. In some embodiments, the viscosity is about 40,000 eps, 41,000 cps, about 42,000 cps, about 43,000 cps, about 44,000 cps, about 45,000 cps, about 46,000 cps, about 47,000 cps, about 48,000 cps, about 49,000 cps, about 50,000 cps, about 51,000 cps, about 52,000 cps, about 53,000 cps, about 54,000 cps, about 55,000 cps, about 56,000 cps, about 57,000 cps, about 58,000 cps, about 59,000 cps, about 60,000 cps, about 61,000 cps, about 62,000 cps, about 63,000 cps, about 64,000 cps, about 65,000 cps, about 66,000 cps, about 67,000 cps, about 68,000 cps, about 69,000 cps, about 70,000 cps, about 71,000 cps, about 72,000 cps, about 73,000 cps, about 74,000 cps, about 75,000 cps, about 80,000 cps, about 90,000 cps, about 95,000 cps, about 100,000 cps, or about 105,000 cps at the time of manufacture, after storage for at least 1 year at room temperature (25° C.), after storage for at least 1.5 years at room temperature, after storage for at least 2 years at room temperature, or after storage for at least 3 years at room temperature.

### Methods of Preparation

Also provided by this disclosure are processes for stabilizing urea for storage that include preparation of any one of the subject formulations (e.g., as described herein), e.g., by dissolving urea in a non-aqueous solvent with a retinoid agent and one or more optionally additional components, combined with a silicone component to provide a stable emulsion composition capable of storage stability.

In some embodiments, the process includes combining:
1% to 30% by weight urea agent selected from urea, hydroxyethyl urea, and combination thereof;
0.01% to 1.5% by weight of a retinoid agent (e.g., as described herein);
20% to 80% by weight of a non-aqueous skin-compatible solvent comprising C(3-6)polyol, ethoxydiglycol, dimethyl ether, or a combination thereof;
4% to 35% silicone agent; and
optionally one or more additional agents.

In some embodiments, the urea agent and non-aqueous skin-compatible solvent are first combined to form a first phase (e.g., dispersed phase) solution (e.g. urea dissolved in the first phase (e.g., dispersed phase) solution, see e.g., “polyol” phase of Tables 1-7. In some embodiments, the first phase (e.g., dispersed phase) solution is combined with a retinoid agent in a silicone/oil-phase (e.g. continuous phase) solution, see e.g., “Silicone/oil phase/continuous phase” of Tables 1-7, to produce storage stable, nonaqueous, emulsion composition of urea. In certain embodiments, the one or more additional agents are combined in a second dispersed phase, before the continuous phase. In some embodiments, the one or more additional components include: 5% to 22% ascorbic acid; 0.5% to 2% ferulic acid; 0.5% to 2% vitamin E; 0.1% to 1% bis-ethylhexyl hydroxydimethoxy benzylmalonate.

In some embodiments, the one or more additional components include: 2% to 7% azelaic acid; 0.1% to 0.5% madecassoside or madecassoside asiaticoside; 0.1% to 1% glycyrrhetinic acid; 0.5% to 1% Pinus pinaster bark extract; 1% to 5% cholesterol ester; 0.5% to 1% bakuchiol.

In some embodiments, the one or more additional components include 5% Azelaic Acid, 0.1% Madecassoside asiaticoside, 0.5% Ferulic Acid, 5% Ascorbic Acid, and Diglycerin and Pinus Pinaster Bark Extract.

Also provided are product storage stable formulations produced by the process according to any one of the embodiments described herein. In some embodiments, the process includes dispersing the dispersed phase components in propanediol. In some embodiments, the process further comprises mixing/agitating the dispersed phase components and propanediol until dissolved and solution is transparent.

After the dispersed phase ingredients are added, the process includes combining the continuous phase ingredients into a closed container and mix until combined. For example, the process includes slowly adding the mixture of the dispersed phases to the continuous phase under high-shear agitation and mix until a uniform, viscous emulsion is formed.

In some embodiments, the component in the continuous phase is a retinoid agent. In some embodiments, the component in the continuous phase is C10-30 Cholesterol/Lanosterol Esters. In some embodiments, the component in the continuous phase is Bakuchiol. In some embodiments, the component in the continuous phase is the silicone agents. In some embodiments, the component in the continuous phase is the silicone and/or oil agents. In some embodiments, the component in the continuous phase is dimethicone and/or dimethicone/PEG-10/15 crosspolymer. In some embodiments, the component in the continuous phase is lauryl PEG-9 polydimethylsilioxyethyl dimethicone. In some embodiments, the component in the continuous phase includes a mixture of allyl methacrylate crosspolymer, polysorbate 20, retinol, and tocopherol. In some embodiments, the components in the continuous phase comprise one or more of (e.g., two or more of, three or more of, four or more of, five or more of, etc.): retinoid agent, C10-30 Cholesterol/Lanosterol Esters, Bakuchiol, silicone and/or oil agents, dimethicone and/or dimethicone/PEG-10/15 crosspolymer, lauryl PEG-9 polydimethylsilioxyethyl dimethicone, allyl methacrylate crosspolymer, polysorbate 20, retinol, and tocopherol. In some embodiments, the components in the continuous phase comprise a combination of two or more of, three or more of, four or more of, five or more of: retinoid agent, C10-30 Cholesterol/Lanosterol Esters, Bakuchiol, silicone and/or oil agents, dimethicone and/or dimethicone/PEG-10/15 crosspolymer, lauryl PEG-9 polydimethylsilioxyethyl dimethicone, allyl methacrylate crosspolymer, polysorbate 20, retinol, and tocopherol. In some embodiments, the components in the continuous phase comprise a: retinoid agent, C10-30 Cholesterol/Lanosterol Esters, Bakuchiol, silicone and/or oil agents, dimethicone and/or dimethicone/PEG-10/15 crosspolymer, lauryl PEG-9 polydimethylsilioxyethyl dimethicone, allyl methacrylate crosspolymer, polysorbate 20, retinol, and tocopherol.

In some embodiments, the components in the dispersed phase comprise one or more of: a non-aqueous solvent (e.g., propanediol), a urea agent, azelaic acid, ascorbic acid, and cinnamic acid (e.g., ferulic acid). In some embodiments, the components in the dispersed phase comprise two or more of, three or more of, four or more of, five or more of: a non-aqueous solvent (e.g., propanediol), a urea agent, azelaic acid, ascorbic acid, and cinnamic acid (e.g., ferulic acid). In some embodiments, the components in the dispersed phase comprise a non-aqueous solvent (e.g., propanediol), a urea agent, azelaic acid, ascorbic acid, and cinnamic acid (e.g., ferulic acid).

In some embodiments, the final emulsion is a slightly yellow, translucent and highly viscous, with a slight ointment-like appearance. In some embodiments, the final emulsion comprises a serum-like texture and consistency. In some embodiments, the final emulsion comprises a serum gel-like texture and consistency. In some embodiments, the final emulsion is a skin serum, such as a facial serum.

### Definitions

The following definitions are set forth to illustrate and define the meaning and scope of the terms used in the description.

It must be noted that as used herein and in the appended claims, the singular forms “a”, “an”, and “the” include plural referents unless the context clearly dictates otherwise. For example, the term “a primer” refers to one or more primers, i.e., a single primer and multiple primers. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

“At least one” means one or more, and also includes individual components as well as mixtures/combinations.

Numbers used in describing quantities of ingredients and/or reaction conditions are to be understood as being modified in all instances by the term “about.” Unless otherwise indicated, percentages and ratios are to be understood as based upon the total weight of the concentrate.

Numerical ranges are meant to include numbers within the recited range, and combinations of subranges between the given ranges. For example, a range from 1-5 includes 1, 2, 3, 4 and 5, as well as subranges such as 2-5, 3-5, 2-3, 2-4, 1-4, etc.

The terms “formulation” and “composition” are used interchangeably herein.

As used herein, the term “non-aqueous” refers to compositions that are substantially anhydrous. Non-limiting examples of substantially anhydrous compositions include, for example, 1% or less water in the subject compositions, such as 0.5% or less, 0.4% or less, 0.3% or less, 0.2% or less, or 0.1% or less water.

It is to be understood that the teachings of this disclosure are not limited to the particular embodiments described, and as such can, of course, vary. It is also to be understood that the terminology used herein is for the purpose of describing particular embodiments only, and is not intended to be limiting, since the scope of the present teachings will be limited only by the appended claims.

The section headings used herein are for organizational purposes only and are not to be construed as limiting the subject matter described in any way. While the present teachings are described in conjunction with various embodiments, it is not intended that the present teachings be limited to such embodiments. On the contrary, the present teachings encompass various alternatives, modifications, and equivalents, as will be appreciated by those of skill in the art.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, some exemplary methods and materials are described herein.

The citation of any publication is for its disclosure prior to the filing date and should not be construed as an admission that the present claims are not entitled to antedate such publication by virtue of prior invention. Further, the dates of publication provided can be different from the actual publication dates which can be independently confirmed. All patents and publications referred to herein are expressly incorporated by reference.

As will be apparent to those of skill in the art upon reading this disclosure, each of the individual embodiments described and illustrated herein has discrete components and features which can be readily separated from or combined with the features of any of the other several embodiments without departing from the scope or spirit of the present teachings. Any recited method can be carried out in the order of events recited or in any other order which is logically possible.

The invention is further defined by reference to the following examples. These examples are representative, and should not be construed to limit the scope of the invention.

### EXAMPLES

### Example 1: Assessment of Formulation Components

A series of experiments were performed to assess and optimize the components of the subject formulations. AA refers to L-ascorbic acid. U refers to urea. % values are wt %.

### Solvents

1,3 propanediol, 1,2 propanediol, butylene glycol, pentylene glycol, and hexanediol were identified as preferred solvents. 1,3 propanediol (trade name: Zemea) is inherently different from to the various polyols described.

1,3-propanediol, sometimes referred to as propanediol, is unique in that it possesses a combination of gentleness on skin (even applied neat, or at 100% concentration), relatively low viscosity (and therefore perceived “lightness” on skin), environmental friendliness (not petroleum-derived), natural derivation (corn or sugar cane), low odor, and moderate ability to solubilize urea.

1,2-propanediol, otherwise referred to in the art as propylene glycol, although of low viscosity and possessing a moderate ability to solubilize urea, is well-known for inducing skin irritation and sensitivity. Additionally, it is derived from petroleum and possesses an unpleasant odor, reminiscent of acetone.

1,3-butanediol, otherwise referred to as butylene glycol, is of low viscosity, possesses a moderate ability to solubilize urea, and is relatively gentle on skin. However, like propylene glycol, it is derived from petroleum and possesses an unpleasant odor, reminiscent of acetone.

also applicable to dipropylene glycol

1,5-pentanediol, otherwise referred to as pentylene glycol, possesses a moderate ability to solubilize urea, low odor, and certain versions are not derived from petroleum but from sugarcane or corn. However, upon application to skin, it imparts a “heavier”, less desirable texture on skin. Additionally, its recommended use level is capped at 5%, limiting usage as a primary solvent.

1,2-hexanediol possesses a moderate ability to solubilize urea. However, upon application to skin, it imparts a “heavier”, less desirable texture on skin, possesses an unpleasant odor reminiscent of acetone, and is derived from petroleum. Additionally, its recommended use level is capped at 10%, limiting usage as a primary solvent.

Glycerin and diglycerin, possess a moderate ability to solubilize urea, are relatively gentle on skin, are low-odor, and are not derived from petroleum. However, they are of a very viscous nature, and impart not only an undesirable, “heavy” texture on skin, but one that is exceedingly sticky.

Dimethyl isosorbide is relatively gentle on skin and not derived from petroleum, and imparts a “light”, not undesirable texture when applied to skin. However, it has a very limited ability to solubilize urea and possesses a slight, but noticeable chemical odor reminiscent of chlorine.

### Urea Agents

In some embodiments, urea is used in the compositions of the present disclosure, for example, for the following reasons:

Urea, when used in sufficient low concentrations (10-15% and below) in leave-on applications, possesses desirable humectant, barrier-repairing and very mild keratolytic properties, which in combination are very effective at improving the feel and look of dry and/or rough skin.

Urea is naturally present not only in the human body but specifically in the skin, where it acts as a natural moisturizing factor (NMF).

Hydroxyethyl urea possesses similar humectant properties, but not the same level of barrier-repairing and mild keratolytic properties of urea.

### Retinoid Agents

In some embodiments, a retinoid agent (e.g., as described herein) is used in the compositions of the present disclosure, for example, for the following reasons:

Retinoid agents (e.g., retinol), when used in sufficient concentrations (e.g., 0.01-1.5% by weight) in leave on applications increases skin production in the lower layers of the skin, resulting in an increase in the speed of desquamation (i.e., the shedding of the upper layer of the skin, or dead skin).

Retinoid agents (e.g., retinol) also has desirable properties, such as boost collogen production, reduce wrinkles, improve skin pigmentation (e.g., reduce skin discoloration), and improve skin pore health.

### Optional Additional Components

Additional ingredients were chosen for their compatibility with (e.g., miscibility in) 1,3 propanediol, 1,2 propanediol, and 1,3 butanediol. Additional notes and observations on each optional additional component are shown below.

### Panthenol (Pro-Vitamin B5)

This is a humectant that shows soothing and moisturizing properties for skin. Both enantiomers, D-panthenol and L-panthenol, are potent humectants. However, only D-panthenol is converted into pantothenic acid in the skin, which confers additional benefits to skin (wound healing, for example).

Research shows that it can reduce irritation to skin by other ingredients

Research also shows barrier-repairing ability (stimulation of physiologic lipid synthesis)

DL-panthenol is a racemic mixture of the two enantiomers; it is in powdered/crystal form.

D-panthenol is a viscous liquid.

DL-panthenol is freely soluble in 1,3 propanediol, 1,2 propanediol and 1,3 propanediol (up to 50%)

D-panthenol is also freely soluble in 1,3 propanediol, 1,2 propanediol and 1,3 propanediol, with no risk of recrystallization at any concentration (as it is already liquid at room temperature).

Inhibition of transepidermal water loss is apparent at concentrations of 1% and above.

### Hyaluronic Acid

Hyaluronic acid is a humectant that shows the ability to form a viscoelastic film on skin that prevents transepidermal water loss.

It is usually incorporated in aqueous solutions in its salt form, sodium hyaluronate

However, there is a raw material blend that is largely free from water, in which it is incorporated in a vehicle of glyceryl polymethacrylate, butylene glycol (1,3 butanediol), and natto gum (trade name Hydrafilm 3MW by The Innovation Company). This makes it compatible with the nonaqueous formulations of the present disclosure.

Documents from The Innovation Company show usage of this material up to 9.1% by weight of the final formula.

The chemical composition is as follows:
75-85% glyceryl polymethacrylate
15-20% butylene glycol
0.5-2% natto gum
0.5-2% hyaluronic acid
   Pinus pinaster Bark Extract

Components of the bark extract of Pinus pinaster species show the ability to recycle vitamin C.

Additionally, there is research to show their general antioxidant, anti-inflammatory and anti-acne properties.

pycnogenol may be used as an alternative when Pinus pinaster bark extract is desired,

a material blend from Kinetik called Pantrofina Skin360 (PS360) is utilized in the subject formulations

PS360, unlike pycnogenol, is already in liquid form as it uses diglycerin as a solvent, making it very easy to incorporate

Additionally, Res Pharma Industriale provides in-vitro and clinical data to show effectiveness against free radical damage, inflammation and acne at a concentration of 0.5% by weight of PS360

The chemical composition is as follows:
90-95% diglycerin
5-10% Pinus pinaster bark extract

### Madecassoside

Centella asiatica extract is often used for its soothing properties.

Madecassoside is a highly purified glycosylated triterpene of Centella asiatica. It is sold by raw material supplier SEPPIC, who share in-vitro and clinical data showing its anti-inflammatory and other effects on skin.

This is a very expensive ingredient ($6.10 per gram), but clinical data from SEPPIC shows desirable ability to reduce erythema (skin redness) in concentrations of 0.2%.

At a concentration of 0.2%, madecassoside is soluble in 1,3 propanediol, 1,2 propanediol and 1,3 butanediol.

In some embodiments, the madecassoside is madecassoside asiaticoside.

### Azelaic Acid

Azelaic acid (AzA) is well studied for its ability to treat acne, rosacea and melasma, due to the fact that it was studied and sold as a prescription drug. Though poorly understood, these effects are believed to be a result of AzA's anti-bacterial, anti-inflammatory, and keratolytic effects, as well as its unique ability to cause apoptosis in abnormal melanocytes.

It is very poorly soluble in most solvents. As a result, all products currently on the market, both prescription and cosmetic, are sold as opaque emulsions, where the AzA is not solubilized but instead finely milled into a powder and suspended in the viscous vehicle.

Because of an inability to solubilize AzA, a preferred component for maximizing delivery into the skin of active ingredients, the team behind prescription product Finacea (currently considered to be the gold standard) chose to manipulate pH, as they discovered that, counterintuitively, a salt form of AzA (formed in aqueous environments in which the pH is higher than the pKa of AzA, 4.15), is slightly better at penetrating skin.

I've discovered that AzA can be solubilized in 1,3 propanediol at relatively high concentrations—up to 10%.

The solubility of AzA in 1,3 propanediol can be slightly increased by the presence of hydroxyethyl urea.

For example, it is possible to solubilize 7.5% AzA with 10% AA, 5% U in a 1,3 propanediol base.

### Ferulic Acid

Ferulic acid is an antioxidant that increases AA's photoprotective effect on skin. It can also somewhat stabilize AA in aqueous systems.

Ferulic acid is readily soluble in 1,3 propanediol, 1,2 propanediol, 1,3 butanediol and dimethyl isosorbide

isosorbide can increase the effectiveness of ferulic acid by enhancing skin penetration.

### Acetyl Zingerone

Acetyl zingerone is a broad-spectrum antioxidant that can prevent lipid peroxidation. It was engineered to be a more stable, more potent derivative of zingerone.

Sytheon provides in-vitro and clinical data showing its antioxidant, photoprotective, and anti-aging properties.

Acetyl zingerone may be used as a replacement for tocopherol.

Acetyl zingerone is readily soluble in 1,3 propanediol, 1,2 propanediol and 1,3 butanediol at the desired concentrations (0.5-1%), eliminating the need for emulsifiers as would be required for tocopherol.

### Glycyrrhizic Acid

Glycyrrhizic acid, like many other derivatives from licorice root (Glycyrrhiza Glabra, Glycyrrhiza Uralensis), shows anti-inflammatory, antioxidant and skin lightening properties.

Unlike 18B-glycyrrhetinic acid, glycyrrhizic acid shows solubility in 1,3-propanediol.

other derivatives of licorice root can be use, such as dipotassium glycyrrhizate, monoammonium glycyrrhizate, etc.

### Isododecane

Isododecane can be used in the present compositions as an additional solvent and/or emollient. In some embodiments, the compositions of the present disclosure include 1% to 20% by weight of isododecane, such as about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% by weight of isododecane. In some embodiments, the compositions of the present disclosure include 7% by weight of isododecane. In some embodiments, the compositions of the present disclosure include 8% by weight of isododecane. In some embodiments, the compositions of the present disclosure include 9% by weight of isododecane. In some embodiments, the compositions of the present disclosure include 10% by weight of isododecane. In some embodiments, the compositions of the present disclosure include 11% by weight of isododecane. In some embodiments, the compositions of the present disclosure include 12% by weight of isododecane. In some embodiments, the compositions of the present disclosure include 13% by weight of isododecane. In some embodiments, the compositions of the present disclosure include 14% by weight of isododecane. In some embodiments, the compositions of the present disclosure include 15% by weight of isododecane.

### Example 2: Exemplary Formulations

The exemplary formulations 1-15 of Tables 1-7, were prepared and assessed as having desirable properties including storage stability.

Key for Tables 1-2
Polyol phase
Silicone/oil phase

TABLE 1
Components of Exemplary Compositions (% by weight)

| Formulation | 1 | 2 | 3 | 4 | 5 |
|---|---|---|---|---|---|
| urea/hydroxy- | 10% | 5% | 10% | 5% | 10% |
| ethyl urea | | | | | |
| C3-C6 polyol | 46% 1,3- | 45.3% | 47.84% | 49.4% 1,3 Propanediol | 56% 1,3 Propanediol |
| | propanediol | 1,3 propanediol | 1,3-propanediol | | |
| silicone | 15% dimethicone | 20% | 15% dimethicone | 10% dimethicone (and) | 10% dimethicone (and) |
| compound(s) | (and) | dimethicone | (and) | Dimethicone/PEG 10/15 | Dimethicone/PEG 10/15 |
| | dimethicone/PEG- | (and) | dimethicone/PEG- | cross polymer (blend of | cross polymer (blend of |
| | 10/15 | dimethicone/PEG- | 10/15 crosspolymer | 77% dimethicone; and | 77% dimethicone; and |
| | crosspolymer | 10/15 | (blend of 77% | 23% dimethicone/PEG- | 23% dimethicone/PEG- |
| | (blend of 77% | crosspolymer | dimethicone; and | 10/15 crosspolymer) | 10/15 crosspolymer) |
| | dimethicone; and | (blend of 77% | 23% | 4% Lauryl PEG-9 | 4% Lauryl PEG-9 |
| | 23% | dimethicone; | dimethicone/PEG- | polydimethylsiloxyethyl | polydimethylsiloxyethyl |
| | dimethicone/PEG- | and 23% | 10/15 | dimethicone | dimethicone |
| | 10/15 | dimethicone/PEG- | crosspolymer) | | |
| | crosspolymer) | 10/15 | 4% lauryl PEG-9 | | |
| | 4% lauryl PEG-9 | crosspolymer) | polydimethylsiloxy- | | |
| | polydimethylsiloxy- | 4% lauryl PEG-9 | ethyl dimethicone | | |
| | ethyl dimethicone | polydimethylsiloxy- | | | |
| | | ethyl dimethicone | | | |
| retinoid agent | 1% retinol | 1% retinol | 1% retinol | 1% retinol complex | 1% retinol complex |
| | complex (trade | complex (trade | complex (trade | (trade name Poly-Pore | (trade name Poly-Pore |
| | name Poly-Pore | name Poly-Pore | name Poly-Pore | 120 TRE) | 120 TRE) |
| | 120 TRE) | 120 TRE) | 120 TRE) | (retinol content about | (retinol content about |
| | (retinol content | (retinol content | (retinol content | 20.5% by weight) | 20.5% by weight) |
| | about 20.5% by | about 20.5% by | about 20.5% by | | |
| | weight) | weight) | weight) | | |
| Additive 1 | 0.5% ferulic acid | 5% azelaic acid | 1% pinus pinaster | 5% azelaic acid | 20% Pentaerythrityl |
| | | | bark extract | | Tetraethylhexanoate |
| | | | (diglycerin and | | |
| | | | pinus pinaster bark | | |
| | | | extract; trade name | | |
| | | | Pantrofina Skin360) | | |
| Additive 2 | 15% ascorbic acid | 0.2% | 5% Pentaerythrityl | 0.5% ascorbic acid | |
| | | madecassoside | Tetraethylhexanoate | | |
| Additive 3 | 8% isododecane | 0.5% ferulic acid | 3% c10-30 | 1% diglycerin and pinus | |
| | | | cholesterol/lanosterol | pinaster bark extract | |
| | | | esters | | |
| Additive 4 | 1% tocopherol | 5% ascorbic acid | 1.64% cholesterol | 0.1% madecassoside; and | |
| | | | | Asiaticoside | |
| | | | | (a blend of about 70% | |
| | | | | madecassoside and about | |
| | | | | 30% asiaticoside by | |
| | | | | weight) | |
| Additive 5 | 0.5% Bis- | 0.5% | 1% ceramide NP | 0.5% Ferulic Acid | |
| | Ethylhexyl | glycyrrhetinic | | | |
| | Hydroxydimethoxy | acid | | | |
| | Benzylmalonate | | | | |
| | (trade name | | | | |
| | Ronacare AP) | | | | |
| Additive 6 | | 0.5% pinus | 0.52% | 10% C12-15 Alkyl | |
| | | pinaster bark | linoleic/linolenic | Benzoate | |
| | | extract | acid (linoleic acid, | | |
| | | (diglycerin and | linolenic acid, | | |
| | | pinus pinaster | tocopherol; trade | | |
| | | bark extract; | name Biosil EFA) | | |
| | | trade name | | | |
| | | Pantrofina | | | |
| | | Skin360) | | | |
| | | (blend of about | | | |
| | | 5-10% pinus | | | |
| | | pinaster bark | | | |
| | | extract and about | | | |
| | | 90-95% | | | |
| | | diglycerin by | | | |
| | | weight) | | | |
| Additive 7 | | C10-30 | 1% bakuchiol | | |
| | | Cholesterol/lano | | | |
| | | sterol esters | | | |
| Additive 8 | | 0.5% bakuchiol | 10% isododecane | | |
| Additive 9 | | 1% tocopherol | | | |
| Additive 10 | | 7% isododecane | | | |

TABLE 2
Components of Exemplary Compositions (% by weight)

| Formulation | 6 | 7 | 8 | 9 | 10 |
|---|---|---|---|---|---|
| urea/hydroxy- | 10% | 10% | 15% | 10% | 5% |
| ethyl urea | | | | | |
| C3-C6 polyol | 48% 1,3-propanediol | 47.83% | 59% 1,3 propanediol | 49% 1,3 | 15% 1,3 |
| | | 1,3 propanediol | | propanediol | propanediol |
| silicone | 5% dimethicone (and) | 7% dimethicone (and) | 20% dimethicone | 30% dimethicone | 40% dimethicone |
| compound(s) | dimethicone/PEG- | dimethicone/PEG-10/15 | (and) | (and) | (and) |
| | 10/15 crosspolymer | crosspolymer (blend of | dimethicone/PEG- | dimethicone/PEG- | dimethicone/PEG- |
| | (blend of 77% | 77% dimethicone; and 23% | 10/15 crosspolymer | 10/15 | 10/15 |
| | dimethicone; and | dimethicone/PEG-10/15 | (blend of 77% | crosspolymer | crosspolymer |
| | 23% | crosspolymer) | dimethicone; and | (blend of 77% | (blend of 77% |
| | dimethicone/PEG- | 5% lauryl PEG-9 | 23% | dimethicone; and | dimethicone; and |
| | 10/15 crosspolymer) | polydimethyl- | dimethicone/PEG- | 23% | 23% |
| | 5% lauryl PEG-9 | siloxyethyl | 10/15 crosspolymer) | dimethicone/PEG- | dimethicone/PEG- |
| | polydimethyl- | dimethicone | 2% lauryl PEG-9 | 10/15 | 10/15 |
| | siloxyethyl | | polydimethyl- | crosspolymer) | crosspolymer) |
| | dimethicone | | siloxyethyl | 2% lauryl PEG-9 | 2% lauryl PEG-9 |
| | | | dimethicone | polydimethyl- | polydimethyl- |
| | | | | siloxyethyl | siloxyethyl |
| | | | | dimethicone | dimethicone |
| | | | | | 34% dimethicone |
| retinoid agent | 1.5% retinol complex | 1.5% retinol complex (trade | 1.5% retinol complex | 1.5% retinol | 1.5% retinol |
| | (trade name Poly-Pore | name Poly-Pore 120 TRE) | (trade name Poly- | complex (trade | complex (trade |
| | 120 TRE) | (retinol content about 20.5% | Pore 120 TRE) | name Poly-Pore | name Poly-Pore |
| | (retinol content about | by weight) | (retinol content | 120 TRE) (retinol | 120 TRE) (retinol |
| | 20.5% by weight) | | about 20.5% by | content about | content about |
| | | | weight) | 20.5% by weight) | 20.5% by weight) |
| Additive 1 | 0.5% ferulic acid | 5% azelaic acid | 4% c10-30 | 4% c10-30 | 4% c10-30 |
| | | | cholesterol/lanosterol | cholesterol/lanosterol | cholesterol/lanosterol |
| | | | esters | esters | esters |
| Additive 2 | 15% ascorbic acid | 0.2% | | 5% isododecane | |
| | | madecassoside/asiaticoside | | | |
| | | (a blend of about 70% | | | |
| | | madecassoside and about 30% | | | |
| | | asiaticoside by weight) | | | |
| Additive 3 | 15% isododecane | 0.5% ferulic acid | | | |
| Additive 4 | 1% tocopherol | 5% ascorbic acid | | | |
| Additive 5 | 0.5% Bis-Ethylhexyl | 0.5% glycyrrhetinic acid | | | |
| | Hydroxydimethoxy | | | | |
| | Benzylmalonate (trade | | | | |
| | name Ronacare AP) | | | | |
| Additive 6 | | 1% pinus pinaster bark extract | | | |
| | | (diglycerin and pinus pinaster | | | |
| | | bark extract; trade name | | | |
| | | Pantrofina Skin360) | | | |
| | | (blend of about 5-10% pinus | | | |
| | | pinaster bark extract and | | | |
| | | about 90-95% diglycerin by | | | |
| | | weight) | | | |
| Additive 7 | | 0.5% bakuchiol | | | |
| Additive 8 | | 1% tocopherol | | | |
| Additive 9 | | 15% isododecane | | | |

TABLE 3
Example Formulation 11

| | | Percent by |
|---|---|---|
| | | weight |
| Phase | Component | (%) |
| Dispersed/polyol phase | Propanediol | 49.4 |
| Dispersed/polyol phase | Urea | 5 |
| Dispersed/polyol phase | Azelaic Acid | 5 |
| Dispersed/polyol phase | Madecass | 0.1 |
| | oside, Asiaticoside | |
| | (a blend of about 70% | |
| | madecassoside and about | |
| | 30% asiaticoside by | |
| | weight) | |
| Dispersed/polyol phase | Ferulic Acid | 0..5 |
| Dispersed/polyol phase | Ascorbic Acid | 5 |
| Dispersed/polyol phase | Diglycerin (and) | 1 |
| | Pinus Pinaster | |
| | Bark Extract | |
| Continuous phase/ | Retinol | 3 |
| Silicone/oil phase | (and) | |
| | Glycine Soja | |
| | (Soybean) Oil | |
| | (retinol content about 10% | |
| | by weight and about 90% | |
| | soybean oil) | |
| Continuous phase/ | C12-15 | 10 |
| Silicone/oil phase | Alkyl Benzoate | |
| Continuous phase/ | Dimethicone (and) | 10 |
| | Dimethicone/PEG- | |
| | 10/15 | |
| Silicone/oil phase | Crosspolymer | |
| | crosspolymer (blend of | |
| | 77% dimethicone; and | |
| | 23% dimethicone/PEG- | |
| | 10/15 crosspolymer) | |
| Continuous phase/ | Lauryl PEG-9 | 4 |
| Silicone/oil phase | Polydimethylsiloxyethyl | |
| | Dimethicone | |
| Continuous phase/ | C10-30 | 4 |
| Silicone/oil phase | Cholesterol/Lano | |
| | Sterol Esters | |
| Continuous phase/ | Tocopherol | 1 |
| Silicone/oil phase | | |
| Continuous phase/ | Bakuchiol | 1 |
| Silicone/oil phase | | |
| Continuous phase/ | Bis-Ethyl | 1 |
| Silicone/oil phase | hexyl | |
| | Hydroxy dimethoxy | |
| | Benzylmalonate | |

TABLE 4
Example Formulation 12

| | | Percent by |
|---|---|---|
| | | weight |
| Phase | Component | (%) |
| Dispersed/polyol | Propanediol | 52.42 |
| phase | | |
| Dispersed/polyol | Urea | 5 |
| phase | | |
| Dispersed/polyol | Ferulic acid | 0.5 |
| phase | | |
| Dispersed/polyol | Azelaic acid | 5 |
| phase | | |
| Continuous | Retinol | 3 |
| phase/ | (and) | |
| Silicone/oil | Glycine Soja | |
| phase | (Soybean) Oil | |
| | (retinol content about 10% | |
| | by weight and about 90% | |
| | soybean oil) | |
| Continuous | pentaerythrityl | 15 |
| phase/ | tetraethylhexanoate | |
| Silicone/oil | | |
| phase | | |
| Continuous | lauryl PEG-9 | 5 |
| phase/ | polydimethylsiloxyethyl | |
| Silicone/oil | dimethicone | |
| phase | | |
| Continuous | c10-30 | 3 |
| phase/ | cholesterol/lanosterol | |
| Silicone/oil | esters | |
| phase | | |
| Continuous | ceramide 3 | 0.5 |
| phase/ | | |
| Silicone/oil | | |
| phase | | |
| Continuous | cholesterol | 0.82 |
| phase/ | | |
| Silicone/oil | | |
| phase | | |
| Continuous | Bakuchiol | 1 |
| phase/ | | |
| Silicone/oil | | |
| phase | | |
| Continuous | linoleic/linolenic acid | 0.26 |
| phase/ | | |
| Silicone/oil | | |
| phase | | |
| Continuous | tocopherol | 0.5 |
| phase/ | | |
| Silicone/oil | | |
| phase | | |
| Continuous | isododecane | 3 |
| phase/ | | |
| Silicone/oil | | |
| phase | | |
| Continuous | Dimethicone and | 3 |
| phase/ | dimethicone/peg-10/15 | |
| Silicone/oil | crosspolymer (blend of | |
| phase | 77% dimethicone; and | |
| | 23% dimethicone/PEG- | |
| | 10/15 crosspolymer) | |

TABLE 5
Example Formulation 13

| | | Percent by |
|---|---|---|
| Phase | Component | weight (%) |
| Dispersed/polyol | Propanediol | 46 |
| phase | | |
| Dispersed/polyol | Urea | 10 |
| phase | | |
| Dispersed/polyol | Ferulic Acid | 0.5 |
| phase | | |
| Dispersed/polyol | Ascorbic Acid | 15 |
| phase | | |
| Continuous | Retinol | 3 |
| phase/ | (and) | |
| | Glycine Soja | |
| Silicone/oil | (Soybean) Oil | |
| phase | (retinol content about 10% | |
| | by weight) | |
| Continuous | isododecane | 8 |
| phase/ | | |
| Silicone/oil | | |
| phase | | |
| Continuous | Dimethicone and | 15 |
| phase/ | dimethicone/peg-10/15 | |
| Silicone/oil | crosspolymer (blend of | |
| phase | 77% dimethicone; and | |
| | 23% dimethicone/PEG- | |
| | 10/15 crosspolymer) | |
| Continuous | lauryl PEG-9 | 4 |
| phase/ | polydimethylsiloxyethyl | |
| Silicone/oil | dimethicone | |
| phase | | |
| Continuous | tocopherol | 1 |
| phase/ | | |
| Silicone/oil | | |
| phase | | |
| Continuous | Bis-Ethylhexyl | 0.5 |
| phase/ | Hydroxydimethoxy | |
| Silicone/oil | Benzylmalonate | |
| phase | | |

TABLE 6
Example Formulation 14

| | | Percent by |
|---|---|---|
| | | weight |
| Phase | Component | (%) |
| Dispersed/polyol | Propanediol | 52.42 |
| phase | | |
| Dispersed/polyol | Urea | 5 |
| phase | | |
| Dispersed/polyol | Ferulic acid | 0.5 |
| phase | | |
| Dispersed/polyol | Azelaic acid | 5 |
| phase | | |
| Continuous | Retinol | 3 |
| phase/ | (and) | |
| Silicone/oil | Glycine Soja | |
| phase | (Soybean) Oil | |
| | (retinol content about 10% | |
| | by weight and about 90% | |
| | soybean oil) | |
| Continuous | pentaerythrityl | 12 |
| phase/ | tetraethylhexanoate | |
| Silicone/oil | | |
| phase | | |
| Continuous | c10-30 | 5 |
| phase/ | cholesterol/lanosterol | |
| Silicone/oil | esters | |
| phase | | |
| Continuous | c10-30 | 2 |
| phase/ | cholesterol/lanosterol | |
| Silicone/oil | esters | |
| phase | | |
| Continuous | ceramide 3 | 0.5 |
| phase/ | | |
| Silicone/oil | | |
| phase | | |
| Continuous | cholesterol | 0.82 |
| phase/ | | |
| Silicone/oil | | |
| phase | | |
| Continuous | Bakuchiol | 1 |
| phase/ | | |
| Silicone/oil | | |
| phase | | |
| Continuous | linoleic/linolenic acid | 0.26 |
| phase/ | | |
| Silicone/oil | | |
| phase | | |
| Continuous | tocopherol | 0.5 |
| phase/ | | |
| Silicone/oil | | |
| phase | | |
| Continuous | isododecane | 5 |
| phase/ | | |
| Silicone/oil | | |
| phase | | |

TABLE 7
Example Formulation 15

| | | Percent by |
|---|---|---|
| | | weight |
| Phase | Component | (%) |
| Dispersed/polyol phase | Propanediol | 56 |
| Dispersed/polyol phase | Urea | 5 |
| Dispersed/polyol phase | Azelaic Acid | 5 |
| Dispersed/polyol phase | Ferulic Acid | 0.5 |
| Dispersed/polyol phase | Ascorbic Acid | 5 |
| Continuous phase/ | Allyl Methacrylate | 1 |
| Silicone/oil phase | Crosspolymer, | |
| | Polysorbate 20, Retinol | |
| | and Tocopherol (retinol | |
| | content about 20.5% by | |
| | weight, and 79.5% of a | |
| | mixture of Allyl | |
| | Methacrylate | |
| | Crosspolymer, | |
| | Polysorbate 20 and | |
| | Tocopherol) | |
| Continuous phase/ | Dimethicone (and) | 20 |
| Silicone/oil phase | Dimethicone/PEG- | |
| | 10/15 | |
| | Crosspolymer | |
| | crosspolymer (blend of | |
| | 77% dimethicone; and | |
| | 23% dimethicone/PEG- | |
| | 10/15 crosspolymer) | |
| Continuous phase/ | Lauryl PEG-9 | 2 |
| Silicone/oil phase | Polydimethylsiloxyethyl | |
| | Dimethicone | |
| Continuous phase/ | C10-30 | 4 |
| Silicone/oil phase | Cholesterol/Lano | |
| | Sterol Esters | |
| Continuous phase/ | Tocopherol | 1 |
| Silicone/oil phase | | |
| Continuous phase/ | Bakuchiol | 0.5 |
| Silicone/oil phase | | |

Creating the anhydrous urea emulsion with the components of any of Table 1-7 includes combining the dispersed phase components with propanediol under agitation, followed by adding the mixture of the dispersed phase components to the continuous phase components under agitation until an emulsion is formed.

### Example 3: Storage Stability

### Compositions

Exemplary compositions were prepared containing approx. 10% urea (e.g., Formulation 15 referred to in Table 7.

Stability of the urea agent can be measured by the amount of ammonia released and/or the odor of ammonia, which are indicators of amount of urea degradation. Stability of the emulsion can be measured by observing changes in viscosity and texture, such as a separation of phases, at various temperatures (5-45° C.) and time periods (1 week to 3 months). Solubility can be measured by the grittiness of the emulsion, as a result of precipitation of urea that is not sufficiently solubilized. Thus, lack of ammonia released and/or ammonia odor is an indicator of strong stability; and lack of grittiness is a result of strong solubility. Tables 8-9 provide the initial stability readings, and Table 10 provides the viscosity and stability testing at various temperatures (5-45° C.) and time periods (1 week to 3 months) Additionally, formulation 15 of Table 7, even when stored at room temperature for 1 year or more, never emitted ammonia odors, indicating the compositions are storage stable.

TABLE 8
Initial Stability readings

| | | | | Viscosity | | |
|---|---|---|---|---|---|---|
| | | | | (spindle: | | |
| | | | | T-E @10 | Sp. | % |
| Color | Odor | Appearance | pH | rpm) | Gr. | Solids |
| Off- | Characteristic | Opaque, | N/A | 200,000 | 1.03 | N/A |
| white | | viscous gel | | cps | | |

TABLE 9
3 cycles- Freeze/Thaw

| | | | | | Viscosity |
|---|---|---|---|---|---|
| | | | | | (spindle: |
| | | | | | T-E@10 |
| Cycle | Color | Odor | Appearance | pH | rpm) |
| 1 | Conforms | Conforms | Conforms | N/A | 113,000 |
| 2 | Conforms | Conforms | Conforms | N/A | 103,000 |
| 3 | Conforms | Conforms | Conforms | N/A | 102,000 |

TABLE 10
Stability conditions

| | | 1 week | 1 month | 2 months | 3 months |
|---|---|---|---|---|---|
| Temperature | date | Jun. 28, 2021 | Jul. 21, 2021 | Aug. 21, 2021 | Sep. 21, 2021 |
| 5° C. | Color | Conforms | Conforms | Conforms | Conforms |
| | Odor | Conforms | Conforms | Conforms | Conforms |
| | Appearance | Conforms | Conforms | Conforms | Conforms |
| | pH | N/A | N/A | N/A | N/A |
| | Viscosity | 96,000 cps | 83,500 cps | 75,500 cps | 74,500 cps |
| 25° C. (RT) | Color | Conforms* | Conforms* | Conforms* | Conforms* |
| | Odor | Conforms | Conforms | Conforms | Conforms |
| | Appearance | Conforms | Conforms | Conforms | Conforms |
| | pH | N/A | N/A | N/A | N/A |
| | Viscosity | 82,000 cps | 66,500 cps | 60,500 cps | 54,500 cps |
| 40° C. | Color | Conforms* | Conforms* | Conforms* | Conforms* |
| w/75% RH | Odor | Conforms | Conforms | Conforms* | Conforms* |
| | Appearance | Conforms | Conforms | Conforms | Conforms |
| | pH | N/A | N/A | N/A | N/A |
| | Viscosity | 73,000 cps | 57,000 cps | 53,500 cps | 51,000 cps |
| 45° C. | Color | Conforms* | Conforms* | Conforms* | Conforms* |
| | Odor | Conforms | Conforms* | Conforms* | Conforms* |
| | Appearance | Conforms | Conforms | Conforms | Conforms |
| | pH | N/A | N/A | N/A | N/A |
| | Viscosity | 70,500 cps | 57,500 cps | 54,000 cps | 53,000 cps |

*slight changes, but still conforms

### Example 4: Clinical Assessment of Formulation 15

Contacting the skin with topical products, such as personal care products, cosmetics and perfumes, may trigger different types of reactions. Adverse reactions can include eczematous contact dermatitis, urticaria, acne and blemishes. In general, the contact dermatitis results from two mechanisms: the primary irritation, through the action of irritant substances; or the sensitization, in the presence of an allergenic ingredient.

In order to evaluate the irritation and sensitization potential of a product, a series of variables must be taken into account: components used in the formulation, ingredient concentration, absorption, amount applied, skin condition, application directions and frequency, as well as the cumulative effect.

Personal care products, household cleaning, cosmetics and perfumes safety studies aim to confirm the absence of risks associated with the product use.

Compatibility studies performed through patch test aim to prove the absence of adverse events during the first contact of a personal care product, household cleaning, cosmetics and perfumes on the skin, proving that the product is safe for use. They consist of repeated applications of the product to the skin, assessing the non-occurrence of irritation or sensitization. The absence of photoirritant or photoallergy potential can also be proved.

This study assessed the effects of Formulation 15 on the skin of patients to prove the absence of the skin primary and cumulative irritation potential and skin sensitization of a Formulation 15 (“Test product” which is a Facial serum that includes Retinol+ACE serum), under maximized conditions, with controlled product quantity and application site, supervised by a dermatologist. Both the investigational product and control were applied to patch test filter paper discs and, then, attached to the right or left back (scapular area) of the study subjects.

Induction Period: The applications were performed on Mondays, Wednesdays and Fridays, during 3 consecutive weeks. Forty-eight hours (48 h), or 72 h (on weekends), after the product application, it was removed by trained technicians and the application site was assessed in order to check the presence of possible clinical signs.

Rest Period: After the induction there was a minimum 10 day-period when no product was applied to the study subjects' back.

Challenge Test: Then, the challenge period started. One single application of the investigational product was carried, followed by readings after 48 h and 72 h of the product being attached to the subjects' dorsum.

The dermatological clinical assessment was made in the beginning and end of the study and the subjects were supervised by a dermatologist throughout the study. Table 11 provides details of the clinical study protocol.

TABLE 11
Clinical study protocol

| | |
|---|---|
| STUDY DURATION | 6 weeks. |
| FREQUENCY OF APPLICATION | 9 applications on the 3 first weeks |
| | (induction period). |
| | 1 application on the last week (challenge |
| | period). |
| APPLICATION SITE | Back (Scapular area). |
| NUMBER OF SUBJECTS | 51 subjects completed the study. |
| POPULATION DESCRIPTION | Female and male subjects, aged from 18 to |
| | 59 years old (mean age: 38 years old), |
| | phototypes II to IV. |
| ETHICS | This study was conducted in conformance |
| | with the Declaration of Helsinki principles, |
| | the applicable regulatory requirements, |
| | including Resolution CNS No. 466/12, in |
| | spirit of the Good Clinical Practices |
| | (Documento de las Americas and ICH E6: |
| | Good Clinical Practice). |
| RESULTS / CONCLUSION | The product did not induce process of skin |
| | sensitization in the study group. According |
| | to this evaluation, the product is considered |
| | safe in the study conditions. The claim |
| | “dermatologically tested” was supported. |

The test product was distributed over the filter paper disc of the patch test, duly identified, with the alphabet letter corresponding to the product. Sterile saline solution (NaCl 0.9%) was used as the control, being distributed on another patch test filter paper disc.

The test product and control were applied always to the same alphabet letter and always attached to the same dorsum area of the subjects throughout the induction period of the study. The test product (in the amount of 0.02 ml) was applied in the concentration of 100%.

The study lasted a total of 6 weeks. The inclusion criteria for the subjects is provided below:
a) Subjects who have cell phone with Internet access with instant messages and voice and video calls applications (WhatsApp);
b) Healthy subjects;
c) Intact skin on test site;
d) Agreement to adhere to the procedures and requirements of the study and to report to the institute on the day(s) and at the time(s) scheduled for the assessments;
e) Ability of giving a consent for his/her participation;
f) Aged from 18 to 70 years old;
g) Phototype (Fitzpatrick): I to IV;
h) Any gender.

The study complied the objective of obtaining, at least, a minimum of 50 responses.

The test product was applied to the study subject's back (scapular area).

### Dermatological Clinical Assessment

A dermatological clinical assessment was performed trough a teleconsultation by a dermatologist through a telephone call or video conference to verify the inclusion and non-inclusion criteria of the study. In addition to the telephone call, the submission of a photograph of the subject's back, the site of application of the products, was requested. The photograph or the video image provided should be done with the aid of a person present in the subject's house at the moment of the teleconsultation, with their previous complete authorization and consent. During the teleconsultation, the confidentiality of the medical attention was ensured, that is, that it was done in a place where only the physician and the authorized person of the technical department were present during the call. Subjects were supervised by a dermatologist throughout the study and assessed in case there were any symptoms or clinical signs.

### Skin Primary and Cumulative Irritation and Sensitization Assessment

The patch test methodology, also known as contact test or epicutaneous test, was used.

Induction Period: The investigational product was always applied to the same duly protected area (right or left back of the subjects). The applications were made three times a week for three consecutive weeks and the product remained in contact with the skin for 48 hours during the week and for 72 hours during weekends.

Rest Period: There was a rest period of, at least, 10 days following the induction period, during which no products were applied.

Challenge period: After the rest period, the test product and control were applied to the right or left back of the subjects on a virgin area, that is, where no products had been applied before.

The product was removed by the trained technicians after approximately 48 hours of contact with the subjects' skin.

The assessments (readings) were performed immediately (48 h reading) and 24 hours (72 h reading) after product removal.

Subjects were instructed to contact the study coordinator at any time, in case they presented any complaints. In these cases, they would be sent for evaluation and guidance by the dermatologist in charge, who would evaluate the subjects, then rate the reaction and follow the appropriate procedure (guidance and/or medication and photographic record, when necessary).

The readings (48 hours after application) were performed soon after the removal of the test product, in all cases in which no clinical signs were observed. If it were observed, the readings would be performed after, at least, 30 minutes and, at most, 60 minutes so that the signs, possibly caused by the removal of the test product, did not represent a false positive result.

### Assessment of Clinical Signs (Readings)

During the study, the areas of product and control applications were evaluated and in case of any clinical sign, it was classified according to a scale recommended by the International Contact Dermatitis Research Group—ICDRG.

TABLE 12

| Scale published by the | |
|---|---|
| International Contact Dermatitis | |
| Research Group - ICDRG | |
| REACTION | RESULT |
| 0 - None | Negative (−) |
| 1 - Mild Erythema | Doubtful (?) |
| 2 - Clear Erythema | Positive (+) |
| 3 - Erythema + Edema + Papules | Positive (++) |
| 4 - Erythema + Edema + Papules + | Positive (+++) |
| Vesicles | |

### Results:

Induction Period: When applied in its pure form (100%), the product caused mild clinical signs on some subjects. For this reason, from the 6th application on, the formulation started being applied at a concentration of 5% and no new clinical signs were observed during the rest of the period.

Challenge Period: No reactions were observed during the challenge phase. The results for this phase indicate the absence of allergenic potential for the product. 11.

The test product (Formulation 15) under the study conditions did not induce process of skin sensitization in the study group. According to this evaluation, the product was considered safe in the study conditions, and thus formulation 15 was “dermatologically tested” and supported.

What is claimed is:

1. A storage stable topical emulsion composition comprising:
a. a dispersed phase that is a homogenous solution comprising:
1% to 30% by weight of urea agent;
dissolved in 10% or more by weight of a non-aqueous skin-compatible solvent selected from polyol, C₍₁₋₆₎ alkanediol, glycol ether, dimethyl ether, and a combination thereof; and
b. a continuous phase comprising:
0.01 to 1.5% of a retinoid agent; and
10% or more by composition weight of a silicone agent selected from cyclic, linear and branched silicones, a silicone crosspolymer, and a combination thereof,
wherein the dispersed phase is immiscible with and contained within the continuous phase.

2. The composition of claim 1, further comprising an antioxidant.

3. The composition of claim 2, wherein the antioxidant is selected from ascorbic acid, and azelaic acid, and any combination thereof.

4. The composition of claim 3, wherein the composition comprises ascorbic acid.

5. The composition of claim 4, wherein the composition comprises 22% or less by weight of ascorbic acid dissolved in the dispersed phase or the continuous phase.

6. The composition of claim 5, wherein the composition comprises about 5% to 15% by weight of ascorbic acid dissolved in the dispersed phase or the continuous phase.

7. The composition of claim 6, wherein the composition comprises about 5% by weight of ascorbic acid dissolved in the dispersed phase or the continuous phase.

8. The composition of any one of claims 3 to 7, wherein the composition comprises azelaic acid.

9. The composition of claim 8, wherein the composition comprises 22% or less by weight of azelaic acid dissolved in the dispersed phase or the continuous phase.

10. The composition of claim 9, wherein the composition comprises about 5% to 15% by weight of azelaic acid dissolved in the dispersed phase or the continuous phase.

11. The composition of claim 10, wherein the composition comprises about 5% by weight of azelaic acid dissolved in the dispersed phase or the continuous phase.

12. The composition of any one of claims 1 to 11, wherein the composition comprises 0.015 to 1% by weight of retinoid agent.

13. The composition of any one of claims 1 to 11, wherein the composition comprises 0.02% by weight of retinoid agent.

14. The composition of any one of claims 1 to 11, wherein the composition comprises 0.5% by weight of ferulic acid.

15. The composition of any one of claims 1 to 14, wherein the retinoid agent is selected from retinol, a retinyl ester, retinal, tretinoin, isotretinoin, alitretinoin, etretinate, acitretin, adapalene, bexarotene and tazarotene.

16. The composition of claim 15, wherein the retinoid agent is retinol.

17. The composition of claim 15, wherein the retinoid agent is a retinyl ester (e.g., retinyl acetate, or retinyl propionate)

18. The composition of claim 15, wherein the retinoid agent is tretinoin.

19. The composition of claim 15, wherein the retinoid is adapalene or tararotene.

20. The composition of claim any one of claims 1 to 19, wherein the continuous phase is a solution of the silicone agent in an oil-phase.

21. The composition of any one of claims 1 to 20, wherein the silicone agent is selected from dimethicone, PEG-10/15 crosspolymer, dimethicone/polyethylene glycol (PEG)-10/15 crosspolymer, lauryl PEG-9 polydimethylsiloxyethyl dimethicone, and a combination thereof.

22. The composition of any one of claims 1-21, wherein the composition comprises 5 to 20% by weight of the silicone agent.

23. The composition of any one of claims 1-22, wherein the urea agent is urea.

24. The composition of any one of claims 1-22, wherein the urea agent is hydroxyethyl urea.

25. The composition of any one of claims 1-24, wherein the urea agent comprises a mixture of urea and hydroxyethyl urea.

26. The composition of any one of claims 1-25, wherein the non-aqueous solvent is selected from 1,3 propanediol, 1,2 propanediol, 1,3 butanediol, 1,5 pentanediol, 1,2 hexanediol, 1,6 hexanediol, glycerol, diglycerol, ethoxydiglycol, dimethyl isosorbide and a combination thereof.

27. The composition of claim 26, wherein the solvent is 1,3 propanediol.

28. The composition of any one of claims 1-27, wherein the composition exhibits a urea degradation rate that is less than the urea degradation rate of a homogenous dispersed phase solution in the absence of the continuous phase emulsion.

29. The composition of any one of claims 1-28, wherein the composition comprises 20% to 80% by weight of the silicone agent of the continuous phase.

30. The composition of any one of claims 1-29, wherein the composition comprises 5% to 20% by weight of the urea agent.

31. The composition of any one of claims 1-30, wherein the composition comprises 0.015% to 0.5% by weight of the retinoid agent.

32. The composition of any one of claims 1-31, wherein the percent by weight ratio of the dispersed phase to the continuous phase in the composition is 19 or less.

33. The composition of any one of claims 1-32, further comprising 10% or less by weight in total of one or more optional additional components dissolved in the dispersed and/or continuous phase (e.g., additional antioxidants, soothing agents, etc.).

34. The composition of claim 33, wherein the one or more optional additional components are selected from tocopherols, tocotrienols (e.g., alpha, beta, delta and gamma tocopherols or alpha, beta, delta and gamma tocotrienols), cinnamic acid, ferulic acid, hydroxy acids (e.g., salicylic acid), panthenol, Pinus pinaster bark extract, hyaluronic acid complex, cholesterol ester, cholesterol, ceramide, linoleic acid, linolenic acid, madecassoside, acetyl zingerone, bakuchiol, bis-ethylhexyl hydroxydimethoxy benzylmalonate, zinc oxide, and titanium dioxide

35. The composition of any one of claims 1-34, wherein the composition comprises about 5% to about 20% by weight of urea.

36. The composition of any one of claim 35, wherein the composition comprises about 5% by weight of urea.

37. The composition of claim 36, wherein the composition comprises about 10% by weight of urea.

38. The composition of any one of claims 33-38, wherein the optional additional component comprises ferulic acid.

39. The composition of claim 34, wherein the composition comprises 0.1% to 2% by weight of ferulic acid.

40. The composition of any one of claims 1 to 39, wherein the composition comprises 0.5% by weight of ferulic acid.

41. The composition of any one of claims 33-40, wherein the optional additional component comprises vitamin E.

42. The composition of claim 41, wherein the vitamin E is selected from alpha, beta, delta and gamma tocopherols and alpha, beta, delta and gamma tocotrienols, and combinations thereof.

43. The composition of claim 41 or 42, wherein the composition comprises 2% or less by weight of vitamin E.

44. The composition of claim 33-43, wherein the optional additional component is bis-ethylhexyl hydroxydimethoxy benzylmalonate.

45. The composition of claim 44, wherein the composition comprises 2% or less by weight of bis-ethylhexyl hydroxydimethoxy benzylmalonate.

46. The composition of any one of claims 33-45, wherein the optional additional component comprises bakuchiol.

47. The composition of claim 46, wherein the composition comprises 2% or less of bakuchiol.

48. The composition of any one of claims 33-47, wherein the optional additional component comprises C10-C30 cholesterol/lanosterol esters.

49. The composition of claim 48, wherein the composition comprises 5% or less of C10-C30 cholesterol/lanosterol esters.

50. The composition of any one of claims 33-49, wherein the optional additional component comprises madecassoside, asiaticoside, or a combination thereof.

51. The composition of claim 50, wherein the composition comprises 1% or less by weight of a combination of madecassoside and asiaticoside.

52. The composition of any one of claims 33-52, wherein the optional additional component comprises glycyrrhetinic acid.

53. The composition of claim 52, wherein the composition comprises 1% or less of glycyrrhetinic acid.

54. The composition of any one of claims 33-53, wherein the optional additional component comprises Pinus pinaster bark extract.

55. The composition of claim 54, wherein the composition comprises 0.5% to 2% by weight of Pinus pinaster bark extract.

56. The composition of any one of claims 33-55, wherein the optional additional component comprises a ceramide.

57. The composition of claim 56, wherein the ceramide is selected from ceramide EOP, ceramide AP, ceramide NG, ceramide NP, ceramide NS, ceramide EOS, ceramide S, ceramide AS, and combinations thereof.

58. The composition of claim 57, wherein the composition comprises 2% or less by weight of ceramide.

59. The composition of any one of claims 33-58, wherein the optional additional component comprises cholesterol.

60. The composition of claim 59, wherein the composition comprises less than 2% by weight of cholesterol.

61. The composition of any one of claims 33-60, wherein the optional additional component comprises a free fatty acid.

62. The composition of claim 61, wherein the free fatty acid is selected from linoleic acid, linolenic acid, stearic acid, palmitic acid, oleic acid, alpha-linoleic, oleic acid, and combinations thereof.

63. The composition of claim 61 or 62, wherein the composition comprises less than 1% free fatty acid.

64. The composition of any one of claims 1 to 63, wherein droplets of the dispersed phase are contained within the continuous phase.

65. The composition of any one of claims 1-64, wherein the composition comprises:
in the dispersed phase:
56% by weight of propanediol;
5% by weight of urea;
5% by weight of azelaic acid; and
5% by weight of ascorbic acid; and
in the continuous phase:
0.02% by weight of the retinoid agent.

66. The composition of any one of claims 1-65, wherein the composition comprises:
in the dispersed phase:
56% by weight of propanediol;
5% by weight of urea;
5% by weight of azelaic acid; and
5% by weight of ascorbic acid; and
in the continuous phase:
1% by weight of a combination of Allyl Methacrylate Crosspolymer, Polysorbate 20, Retinol and Tocopherol;
4% by weight of C10-30 cholesterol/lanosterol esters;
0.5% by weight of Bakuchiol;
1% by weight of Tocopherol; and
20% by weight of dimethicone and dimethicone/PEG-10/15 crosspolymer.

67. The composition of any one of claims 1-65, wherein the composition comprises:
in the dispersed phase:
56% by weight of propanediol;
5% by weight of urea;
5% by weight of azelaic acid; and
5% by weight of ascorbic acid; and
in the continuous phase:
0.2% by weight of the retinoid agent;
0.8% by weight of a combination of Allyl Methacrylate Crosspolymer, Polysorbate 20, and Tocopherol;
4% by weight of C10-30 cholesterol/lanosterol esters;
0.5% by weight of Bakuchiol;
1% by weight of Tocopherol; and
20% by weight of dimethicone and dimethicone/PEG-10/15 crosspolymer.

68. A ready-to-use topical preparation in a multi-use container which is pre-filled with a storage stable topical composition according to any of the claims 1-67, wherein the multi-use container comprises means for dispensing a single dose of the storage stable topical composition.

69. The preparation of claim 68, wherein the multi-use container is a time-release container that delivers urea and the retinoid agent consistently over a period of time.

70. The preparation of claim 69, wherein the time-release container does not release urea and the retinoid agent all at once.

71. The preparation of any one of claims 68-70, wherein the storage stable topical composition is sealed in the container.

72. The preparation of any one of claims 68-63, wherein the container is placed in packaging.

73. A process for producing a storage stable emulsion composition for topical application, the process comprising:
a) combining:
i. 1% to 30% by weight of urea agent selected from urea, hydroxyethyl urea, and combination thereof;
ii. 10% to 80% by weight of a non-aqueous skin-compatible solvent selected from polyol, C₍₁₋₆₎ alkanediol, glycol ether, dimethyl ether, and a combination thereof; and
iii. optionally one or more additional agents;
thereby dissolving the urea agent, and one or more additional agents in the non-aqueous solvent to produce a dispersed phase that is a homogenous solution; and
b) suspending the dispersed phase in a continuous phase comprising 10% or more by weight of a silicone compound dissolved in an oil-phase solution; and 0.01% to 1.5% of a retinoid agent;
to produce an emulsion composition that is storage stable.

74. The process of claim 73, wherein the silicone compound is selected from cyclic, linear and branched silicones, a silicone crosspolymer, and a combination thereof.

75. The process of claim 73 or 74, wherein the one or more additional agents comprise ascorbic acid.

76. The process of claim 75, wherein the dispersed phase comprises 22% or less by weight of ascorbic acid.

77. The process of claim 76, wherein the dispersed phase comprises about 5% to about 15% by weight of ascorbic acid.

78. The process of any one of claims 73-77, wherein the one or more additional agents comprise ferulic acid.

79. The process of claim 78, wherein the composition comprises 2% or less by weight of ferulic acid.

80. The process of any one of claims 73-79, wherein the one or more additional agents comprise:
0.5% to 2% ferulic acid; and
0.5% to 2% Pinus pinaster bark extract.

81. The process of claim 73-80, wherein the one or more additional agents comprise about 3% to about 10% by weight azelaic acid.

82. The process of any one of claims 73-81, wherein the continuous phase further comprises a lipid component.

83. The process of claim 82, wherein the lipid component is selected from cholesterol, ceramides, free fatty acids, and combinations thereof.

84. The process of any one of claims 73-82, wherein the continuous phase prevents or reduces precipitation of urea out of the emulsion composition.

85. The process of any one of claims 73-84, wherein the urea agent, prior to dissolving in the non-aqueous solvent, is a crystalline form of the urea agent with a particle size of about 100 m or more.

86. The process of claim 85, wherein suspending the dispersed phase in the continuous phase prevents or reduces recrystallization of urea from the dispersed phase.

87. The process of claim 86, wherein said suspending comprises suspending droplets of the dispersed phase within the continuous phase.

88. A product produced by the process according to any one of claims 73-87.

89. The product of claim 88, wherein the product is a serum.
